SEQUENCE LISTING (14 sequences)

SEQ ID NO: 1 - DNA, 18 bp, Artificial Sequence
  Synthetic
gtgctgcgccaagaaaat

SEQ ID NO: 2 - DNA, 20 bp, Artificial Sequence
  Synthetic
tcaactcgcagacacgactc

SEQ ID NO: 3 - DNA, 20 bp, Artificial Sequence
  Synthetic
tacaacctgaccatcgcttc

SEQ ID NO: 4 - DNA, 20 bp, Artificial Sequence
  Synthetic
gcccccagagacttgttgta

SEQ ID NO: 5 - DNA, 20 bp, Artificial Sequence
  Synthetic
ccctccaacaacagcaagga

SEQ ID NO: 6 - DNA, 19 bp, Artificial Sequence
  Synthetic
acccaacagggcatccaag

SEQ ID NO: 7 - DNA, 22 bp, Artificial Sequence
  Synthetic
catcaccacacctgaaagcaga

SEQ ID NO: 8 - DNA, 20 bp, Artificial Sequence
  Synthetic
tggacagtgtcccgaaggag

SEQ ID NO: 9 - DNA, 23 bp, Artificial Sequence
  Synthetic
caaccaaagacgctggaactatt

SEQ ID NO: 10 - DNA, 22 bp, Artificial Sequence
  Synthetic
ccctcttatccaggccataagc

SEQ ID NO: 11 - DNA, 21 bp, Artificial Sequence
  Synthetic
tcagagacatgcttcgacgaa

SEQ ID NO: 12 - DNA, 19 bp, Artificial Sequence
  Synthetic
ctgaccgaagcgttgctgt

SEQ ID NO: 13 - DNA, 18 bp, Artificial Sequence
  Synthetic
tccactggcgtcttcacc

SEQ ID NO: 14 - DNA, 21 bp, Artificial Sequence
  Synthetic
ggcagagatgatgaccctttt

(19) United States
(12) Patent Application Publication
(10) Pub. No.: US 2024/0033236 A1
(43) Pub. Date: Feb. 1, 2024

(54) METHODS OF TREATING HERPES VIRAL INFECTION WITH 4-PHENYLBUTYRATE (PBA) OR A PHARMACEUTICALLY ACCEPTABLE SALT THEREOF

(71) Applicant: THE BOARD OF TRUSTEES OF THE UNIVERSITY OF ILLINOIS, Urbana, IL (US)

(72) Inventors: Tejabhiram Yadavalli, Chicago, IL (US); Deepak Shukla, Chicago, IL (US)

(21) Appl. No.: 18/030,139

(22) PCT Filed: Oct. 13, 2021
(86) PCT No.: PCT/US2021/054868

§ 371 Date: Apr. 4, 2023

Related U.S. Application Data
(60) Provisional application No. 63/091,236, filed on Oct. 13, 2020.

(51) Int. Cl. A61K 31/192; A61P 31/22; A61K 31/522; A61K 31/7072; A61K 9/00; A61K 45/06

(52) U.S. Cl. CPC A61K 31/192; A61P 31/22; A61K 31/522; A61K 31/7072; A61K 9/0048; A61K 45/06

(57) ABSTRACT

Disclosed herein are compositions comprising: 4-phenylbutyrate (PBA) or a pharmaceutically acceptable salt thereof in an effective concentration as a therapeutic or prophylactic agent against viral infections, e.g., herpes simplex vims (HSV) infections. Also provided are methods for treating a disease or disorder in a subject, such as a viral infection (e.g., HSV infection), comprising administering to the subject a therapeutically or prophylactically effective amount of 4-phenylbutyrate (PBA) or a pharmaceutically acceptable salt thereof.

### CROSS REFERENCE TO RELATED APPLICATIONS

This application claims the benefit of U.S. Provisional Patent Application No. 63/091,236, filed on Oct. 13, 2020, which is incorporated herein by reference in its entirety.

### BACKGROUND OF THE DISCLOSURE

Herpes simplex viruses (HSV) are among the most ubiquitous pathogens with global seroprevalence in the range of 90%. Recurrent infections lead to painful ulcerative keratitis and lesions in the eye and genitalia in latently infected individuals. Currently there is only one approved treatment, acyclovir, which acts via the inhibition of a single aspect of viral life cycle, viral DNA replication. Among other cellular pathways tightly regulated by the virus during its life cycle is the unfolded protein response to endoplasmic reticulum (ER) stress. Previous studies have shown that the pharmacological modulation of this pathway through inducers and alleviators of ER stress results in the inhibition of viral replication.

### SUMMARY OF THE DISCLOSURE

In accordance with the principles herein, 4-phenylbutyrate (PBA), a chemical chaperone based potent alleviator of ER stress, was tested for its effects on HSV-1 infection. Through in vitro studies it was proven that application of PBA to HSV infected cells resulted in the down-regulation of a pro-viral, ER localized host protein CREB3 and resulted in the inhibition of viral protein synthesis. Subsequently, systemic treatment with PBA in murine models of ocular and genital HSV infection resulted in equal or greater viral inhibition than acyclovir. Thus, PBA can be used as an alternative to current antivirals to treat both ocular HSV-1 and genital HSV-2 infections and is a candidate for future human trials.

Thus, in accordance with the principles herein, an exemplary composition can comprise: 4-phenylbutyrate (PBA) or a pharmaceutically acceptable salt thereof in an effective concentration as a therapeutic agent against viral infections. An exemplary therapeutic agent against viral infections inhibits virus replication in a cell, reduces viral spread or reduces the severity of infection. A therapeutic agent against viral infections may also prevent viral infection.

The PBA in the composition can be a small molecule drug. In some cases, the PBA is present as a salt. In some cases, the PBA is sodium 4-phenylbutanoate. The concentration of the PBA or salt thereof can range from about 1 mM to about 200 mM, or has a cumulative concentration ranging from about 10 mg/kg to about 600 mg/kg. In some cases, the composition comprises 100 mg/kg of the PBA or salt thereof.

The PBA can be encapsulated in a drug delivery agent. In some cases, the composition further comprises a drug delivery agent. The drug delivery agent can be selected from a polymeric agent, activated carbon agent, metal agent, metal oxide agent, liposomal agent, peptide/protein agent, sugar agent, and combinations thereof. The therapeutic and drug delivery agent can be in nanometer, micrometer or macro form. In some cases, the composition comprises PBA and a drug delivery agent as a nanoformulation, microformulation, or macroformulation.

The composition can include PBA combined with another therapeutic agent. In some cases, the composition further comprises one or more additional therapeutic agents. The one or more additional therapeutic agent can be selected from an antiviral agent, anti-inflammatory agent, pain reducing agent, and combinations thereof.

The antiviral agent can be Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine or sofobuvir. In some cases, the composition comprises 10 mg/kg of the antiviral agent.

In some cases, the composition is formulated for ophthalmic administration. In some cases, the composition comprises an ophthalmic excipient. In some cases, the ophthalmic excipient is selected from Cyclodextrins, Carbopol or carbomer or acrylic acid polymers, Poloxamers, Xyloglucan, Methylcellulose, Hydroxypropyl Methylcellulose, Ethyl (Hydroxyethyl) Cellulose, Pseudolatexes, Cellulose Acetate Phthalate, Gellan Gum, Alginate, Carrageenans, Hyaluronic Acid, Sodium acetate, Edetate disodium, Hypromellose, Acetic acid, Alcohol, Alginic acid, Amerchol-cab, Antipyrine, Benzalkonium chloride, Benzododecinium bromide, Boric acid, Caffeine, Calcium chloride, Carbomer 1342, Carbomer 934P, Carbomer 940, Carbomer homopolymer type B (allyl pentaerythritol cross-linked), Carboxymethylcellulose sodium, Castor oil, Cetyl alcohol, Chlorobutanol, Citric acid, Citric acid monohydrate, Creatinine, Divinylbenzene styrene copolymer, Ethylene vinyl acetate copolymer, Gellan gum (low acyl), Glycerin, Glyceryl stearate, Hypromelloses, Lanolin, Lauralkonium chloride, Lauroyl sarcosine, Magnesium chloride, Methylparaben, Mineral oil, Nonoxynol-9, Octoxynol-40, Petrolatum, Phenylethyl alcohol, Phenylmercuric acetate, Phenylmercuric nitrate, Polidronium chloride, Poloxamer 188 or 407, Polycarbophil, Polyethylene glycol 400 or 8000, Polyoxyl 35 castor oil, Polyoxyl 40 hydrogenated castor oil, Polyoxyl 40 stearate, Polypropylene glycol, Polysorbate 20, Polyvinyl alcohol, Potassium chloride, Potassium sorbate, Povidone K29/32, Povidone K30, Povidone K90, Povidones, Propylene glycol, Propylparaben, Soda ash, Sodium acetate, Sodium bisulfate, Sodium borate, Sodium borate decahydrate, Sodium carbonate, Sodium chloride, Sodium citrate, Sodium metabisulfite, Sodium nitrate, Sodium sulfate, Sodium sulfite, Sodium thiosulfate, Sorbic acid, Sorbitol, Stabilized oxychloro complex, Sulfuric acid, Thimerosal, Titanium dioxide, Tocophersolan, Trisodium citrate dehydrate, Tromethamine, Tyloxapol, Xanthan gum, Zinc chloride, and combinations thereof.

In some cases, the composition is formulated for topical administration. In some cases, the composition comprises a mucosal penetrating agent, such as poloxamer 407.

The disclosure provides for methods of treating a disease or disorder in a subject comprising administering any of the compositions described herein. In addition, the disclosure provides for use of any of the compositions described herein to prepare a medicament for treating a disease or disorder in a subject.

Also provided herein are methods of treating a disease or disorder in a subject, comprising administering to the subject a therapeutically effective amount of 4-phenylbutyrate (PBA) or a pharmaceutically acceptable salt thereof. In some cases, the disease or disorder is a viral infection. In some cases, the viral infection is caused by Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, humanherpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

In some cases, the disease or disorder is associated with a herpes simplex virus (HSV). In some cases, the disease or disorder is associated with herpes simplex virus 1 (HSV-1) or herpes simplex virus 2 (HSV-2). In some cases, the disease or disorder is corneal inflammation, corneal hypertension, corneal sensitivity, corneal dryness, keratitis, ocular herpes, herpes gingivostomatitis, herpes labialis, herpes genitalis, herpetic whitlow, herpes gladiatorum, herpesviral encephalitis, herpesviral meningitis; herpes esophagitis, neonatal herpes simplex, herpetic sycosis, eczema herpeticum, or herpetic keratoconjunctivitis.

In some cases, the method comprises administering to a patient a therapeutically effective amount of a composition comprising PBA or a pharmaceutically acceptable salt thereof, and one or more additional therapeutic agents. In some cases, the one or more additional therapeutic agent is selected from an antiviral agent, anti-inflammatory agent, pain reducing agent, and combinations thereof. In some cases, the antiviral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine, or sofobuvir.

An exemplary method of treating a viral infection is set forth. The method can comprising administering PBA in an amount that inhibits virus replication in a cell or reduces viral spread.

The method can include the step of administering an effective dosage to reduce the severity of an infection caused by one or more of Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, humanherpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV and Venezuelan equine encephalomyelitis VEE. The method can include the step of PBA administration and a combined therapeutic agent or drug delivery system administered via parenteral, nasal, oral, pulmonary, topical, vaginal, rectal ocular or sublingual route. In some cases, the method comprises parenteral, nasal, oral, pulmonary, topical, vaginal, rectal, ophthalmic, or sublingual administration.

The method can include administering PBA, any of the disclosed composition or therapeutic agents via prophylactic or therapeutic administration. The method can include administering the PBA, composition or therapeutic agent via a preloaded syringe, or a preloaded dermal (skin) patch. In some cases, the method comprises prophylactic or therapeutic administration. In some cases, the method comprises administration via preloaded syringe. In some cases, the method comprises administration via preloaded dermal (skin) patch.

An exemplary ophthalmic composition can comprise: PBA and one or more of pharmaceutically acceptable ophthalmic excipients. The pharmaceutically acceptable ophthalmic excipients can be selected from Cyclodextrins, Carbopol or carbomer or acrylic acid polymers, Poloxamers, Xyloglucan, Methylcellulose, Hydroxypropyl Methylcellulose, Ethyl (Hydroxyethyl) Cellulose, Pseudolatexes, Cellulose Acetate Phthalate, Gellan Gum, Alginate, Carrageenans, Hyaluronic Acid, Sodium acetate, Edetate disodium, Hypromellose, Acetic acid, Alcohol, Alginic acid, Amerchol-cab, Antipyrine, Benzalkonium chloride, Benzododecinium bromide, Boric acid, Caffeine, Calcium chloride, Carbomer 1342, Carbomer 934P, Carbomer 940, Carbomer homopolymer type B (allyl pentaerythritol cross-linked), Carboxymethylcellulose sodium, Castor oil, Cetyl alcohol, Chlorobutanol, Citric acid, Citric acid monohydrate, Creatinine, Divinylbenzene styrene copolymer, Ethylene vinyl acetate copolymer, Gellan gum (low acyl), Glycerin, Glyceryl stearate, Hypromelloses, Lanolin, Lauralkonium chloride, Lauroyl sarcosine, Magnesium chloride, Methylparaben, Mineral oil, Nonoxynol-9, Octoxynol-40, Petrolatum, Phenylethyl alcohol, Phenylmercuric acetate, Phenylmercuric nitrate, Polidronium chloride, Poloxamer 188 or 407, Polycarbophil, Polyethylene glycol 400 or 8000, Polyoxyl 35 castor oil, Polyoxyl 40 hydrogenated castor oil, Polyoxyl 40 stearate, Polypropylene glycol, Polysorbate 20, Polyvinyl alcohol, Potassium chloride, Potassium sorbate, Povidone K29/32, Povidone K30, Povidone K90, Povidones, Propylene glycol, Propylparaben, Soda ash, Sodium acetate, Sodium bisulfate, Sodium borate, Sodium borate decahydrate, Sodium carbonate, Sodium chloride, Sodium citrate, Sodium metabisulfite, Sodium nitrate, Sodium sulfate, Sodium sulfite, Sodium thiosulfate, Sorbic acid, Sorbitol, Stabilized oxychloro complex, Sulfuric acid, Thimerosal, Titanium dioxide, Tocophersolan, Trisodium citrate dehydrate, Tromethamine, Tyloxapol, Xanthan gum, Zinc chloride, or a combination thereof.

The composition can be provided in a suitable carrier and/or is formulated for topical administration. In some cases, the composition comprises a mucosal penetrating agent. In some cases, the mucosal penetrating agent is poloxamer 407.

Also provided are methods of treating an ocular disease or condition in a subject in need thereof, comprising administering to the subject 4-phenylbutyrate (PBA) or a pharmaceutically acceptable salt thereof. In some cases, the method comprises administering to a patient a therapeutically effective amount of a composition comprising PBA or a pharmaceutically acceptable salt thereof, and one or more additional therapeutic agents. In some cases, the ocular disease or condition is corneal inflammation, corneal hypertension, corneal sensitivity, corneal dryness, ocular herpes, herpetic keratoconjunctivitis, or keratitis.

An exemplary method of treating an ocular disease or condition in a subject in need can comprise administering to the subject an effective amount of a PBA composition. The method can further comprise the step of forming the composition to include mixing approximately 10 mg/kg of an antiviral agent with approximately 100 mg/kg of PBA.

Also provided herein are compositions for treating a disease or disorder in a subject, wherein the composition comprises a therapeutically effective amount of 4-phenylbutyrate (PBA) or a pharmaceutically acceptable salt thereof. In some cases, the disease or disorder is a viral infection. In some cases, the viral infection is caused by Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, humanherpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

In some compositions for treating a disease or disorder, the disease or disorder is associated with a herpes simplex virus (HSV). In some cases, the disease or disorder is associated with herpes simplex virus 1 (HSV-1) or herpes simplex virus 2 (HSV-2). In some cases, the disease or disorder is corneal inflammation, corneal hypertension, corneal sensitivity, corneal dryness, keratitis, ocular herpes, herpes gingivostomatitis, herpes labialis, herpes genitalis, herpetic whitlow, herpes giadiatorum, herpesviral encephalitis, herpesviral meningitis, herpes esophagitis, neonatal herpes simplex, herpetic sycosis, eczema herpeticum, or herpetic keratoconjunctivitis.

In some cases, the composition for treating a disease or disorder comprises a therapeutically effective amount of PBA or a pharmaceutically acceptable salt thereof, and one or more additional therapeutic agents. In some cases, the one or more additional therapeutic agent is selected from an antiviral agent, anti-inflammatory agent, pain reducing agent, and combinations thereof. In some cases, the antiviral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine, or sofobuvir.

In addition, compositions for treating a viral infection comprising a therapeutically effective amount of 4-phenylbutyrate (PBA) or a pharmaceutically acceptable salt thereof are provided herein. For example, the composition can comprise PBA in an amount that inhibits virus replication in a cell or reduces viral spread.

For example, the composition for treating a viral infection comprises a dosage of PBA that effectively reduces the severity of an infection caused by one or more of Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, humanherpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACY, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV and Venezuelan equine encephalomyelitis VEE. In some cases, the composition for treating a viral infection comprises PBA or a therapeutically acceptable salt thereof and a combined therapeutic agent or drug delivery system, wherein the composition is formulated for administration via parenteral, nasal, oral, pulmonary, topical, vaginal, rectal ocular or sublingual route. In some cases, the composition is formulation for parenteral, nasal, oral, pulmonary, topical, vaginal, rectal, ophthalmic, or sublingual administration.

The composition for treating a viral infection can be a composition that is administered prophylactically or the composition can be administered therapeutically. The composition can be formulated for administering the PBA, composition or therapeutic agent via a preloaded syringe, or a preloaded dermal (skin) patch.

Also provided are compositions for treating an ocular disease or condition in a subject in need thereof, comprising 4-phenylbutyrate (PBA) or a pharmaceutically acceptable salt thereof. For example, the composition comprises 4-phenylbutyrate (PBA) or a pharmaceutically acceptable salt thereof and an acceptable ophthalmic excipient. In some cases, the composition for treating an ocular disease or condition comprises PBA or a pharmaceutically acceptable salt thereof, and one or more additional therapeutic agents. In some cases, the ocular disease or condition is corneal inflammation, corneal hypertension, corneal sensitivity, corneal dryness, ocular herpes, herpetic keratoconjunctivitis, or keratitis.

An exemplary composition for treating an ocular disease or condition in a subject in need can comprise an effective amount of a PBA composition. The composition be formed by mixing approximately 10 mg/kg of an antiviral agent with approximately 100 mg/kg of PBA.

Also provided herein are uses of 4-phenylbutyrate (PBA) or a pharmaceutically acceptable salt thereof for the preparation of a medicament for treating a disease or disorder in a subject. In some cases, the disease or disorder is a viral infection. In some cases, the viral infection is caused by Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, humanherpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

In some cases, the disease or disorder is associated with a herpes simplex virus (HSV). In some cases, the disease or disorder is associated with herpes simplex virus 1 (HSV-1) or herpes simplex virus 2 (HSV-2). In some cases, the disease or disorder is corneal inflammation, corneal hypertension, corneal sensitivity, corneal dryness, keratitis, ocular herpes, herpes gingivostomatitis, herpes labialis, herpes genitalis, herpetic whitlow, herpes gladiatorurn, herpesvirai encephalitis, herpesviral meningitis, herpes esophagitis, neonatal herpes simplex, herpetic sycosis, eczema herpeticum, or herpetic keratoconjunctivitis.

In some uses, the medicament comprises a therapeutically effective amount of a composition comprising PBA or a pharmaceutically acceptable salt thereof, and one or more additional therapeutic agents. In some cases, the one or more additional therapeutic agent is selected from an antiviral agent, anti-inflammatory agent, pain reducing agent, and combinations thereof. In some cases, the antiviral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine, or sofobuvir.

Also provided are uses of 4-phenylbutyrate (PBA) or a pharmaceutically acceptable salt thereof for the preparation of a medicament for treating a viral infection. The medicament can comprise PBA in an amount that inhibits virus replication in a cell or reduces viral spread.

In any of the uses, the medicament comprises an effective dosage of 4-phenylbutyrate (PBA) or a pharmaceutically acceptable salt thereof effective to reduce the severity of an infection caused by one or more of Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, humanherpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV and Venezuelan equine encephalomyelitis VEE. The medicament comprises and a combined therapeutic agent or drug delivery system, and the medicament, PBA, therapeutic agent or drug delivery system is formulated for parenteral, nasal, oral, pulmonary, topical, vaginal, rectal ocular or sublingual route. In some cases, the method comprises parenteral, nasal, oral, pulmonary, topical, vaginal, rectal, ophthalmic, or sublingual administration.

In any of the disclosed uses, the PBA or the medicament is administered prophylactically or the medicament is administered therapeutically. In any of the uses, the medicament, PBA, or therapeutic agent can be administered via a preloaded syringe, or a preloaded dermal (skin) patch.

In any of the disclosed uses, the PBA, composition or medicament can be provided in a suitable carrier and/or is formulated for topical administration. In some uses, the PBS, composition or medicament comprises a mucosal penetrating agent. In some cases, the mucosal penetrating agent is poloxamer 407.

Also provided are uses of 4-phenylbutyrate (PBA) or a pharmaceutically acceptable salt thereof for the preparation of a medicament for treating an ocular disease or condition in a subject in need thereof. For example, the medicament comprises—phenylbutyrate (PBA) or a pharmaceutically acceptable salt thereof and an acceptable ophthalmic excipient. In some cases, the medicament comprises a therapeutically effective amount of a composition comprising PBA or a pharmaceutically acceptable salt thereof, and one or more additional therapeutic agents. In some cases, the ocular disease or condition is corneal inflammation, corneal hypertension, corneal sensitivity, corneal dryness, ocular herpes, herpetic keratoconjunctivitis, or keratitis.

In an exemplary use, the medicament comprises an effective amount of a PBA composition, and the composition is formed by mixing approximately 10 mg/kg of an antiviral agent with approximately 100 mg/kg of PBA.

Other embodiments constructed in accordance with the principles of the present disclosure are contemplated herein as well.

### BRIEF DESCRIPTION OF THE DRAWINGS

FIGS. 1A-1L show that CREB3 is upregulated upon HSV-1 infection. FIGS. 1A and 1C show immunoblots for the shown proteins of human corneal epithelial cells that were either mock infected or infected with HSV-1. FIG. 1B shows cells fixed with 4% PFA and stained for CREB3 (Red), HSV-1 (Green) and nucleus (blue). FIG. 1D show immunoblotting analysis and FIG. 1E shows quantitative reverse transcription PCR of HCEs infected with 0.1 MOI HSV-1 17 GFP for 0, 3, 6, 12, 24 or 48 hours. FIG. 1F shows qRT-PCR of downstream effectors of CREB3 in HSV-1 infected HCEs, and FIG. 1G shows fluorescent imaging of the same. FIG. 1H shows immunoblotting and FIG. 1I shows imaging for CREB3 of primary human corneal epithelial cells infected with HSV-1. FIG. 1J shows a gene ontology diagram of CREB3 showing its role in unfolded protein response and negative regulation of ER-stress induced apoptosis. FIG. 1K shows HCEs that were transfected with GFP or HPSE-GFP overexpression plasmid for 24 hours, prior to the addition of mock or 0.1 MOI HSV-1 Kos. The cells were collected 24 hpi and immunoblotted for shown proteins. Transfection efficiency and expression of HPSE-GFP can be seen via fluorescent images in FIG. 1L.

FIGS. 2A-2G show that modulation of CREB3 expression affects viral life cycle: FIG. 2A shows fluorescent microscope imaging of HCEs transfected with CREB3 siRNA and treated with HSV-1 17 GFP 24 hours post infection. FIG. 2B shows cell lysates and supernatant from the cell culture used as inoculum for a plaque assay on Vero cells, FIG. 2C shows immunoblotting analysis of cell lysates, and FIG. 2D shows a flow cytometry analysis. FIG. 2E shows HCEs transfected with increasing concentrations of CREB3 siRNA and immunoblotted to evaluate changes in ATF4 and CHOP along with GAPDH as the loading control. FIG. 2F shows immunoblotting of HCEs transfected with CREB3 overexpression plasmid and treated with HSV-1 17 GFP 24 hours post infection. FIG. 2G shows cell culture supernatant was collected and used as inoculum to assess released virus via plaque assay on Vero cells.

FIGS. 3A-3G show that ER stress alleviation through PBA reduces viral infection and induces CHOP and ATF expression. HCEs were infected with HSV-1 K26RFP and treated with tunicamycin (1 μM), thapsigargin (1 μM), Brefeldin-A (1 μM), salubrinal (75 μM) or PBA (10 mM). FIG. 3A shows fluorescent imaging for HSV-1, and FIG. 3B shows lysed cells used as inoculum on Vero cells to assess viral titer via plaque assay. FIG. 3C shows HCE viability in the presence of various concentration of PBA under non-infectious and infectious conditions as determined via MTT assay. FIG. 3D shows the effect of PBA on viral entry as assessed through a 8-galactosidase producing reporter HSV-1 gL86 using ONPG as the substrate. FIG. 3E shows concentration dependent viral inhibition via a plaque assay of HCEs infected and treated with PBA with various concentrations. FIG. 3F shows HCEs treated with Mock DMSO or PBA (10 mM) for 24 hours prior to fixing and staining with fluorescently labelled antibodies. FIG. 3G shows immunoblotting for shown proteins for HCEs infected in the presence or absence of PBA.

FIGS. 4A-4I show therapeutic efficacy of PBA in ex vivo human and in vivo murine tissues. FIG. 4A shows stereoscopic images of human corneas infected with HSV-1, and FIG. 4B shows plaque assay titration results for amount of virus in the corneas. FIG. 4C shows fluorescent microscopy images of primary human corneal epithelial cells infected with HSV-1 17 GFP followed by treatment with PBA (10 mM) or DMSO control. FIG. 4D shows immunoblots of cell lysates to detect the presence of viral proteins. FIG. 4E shows fluorescent images of skin epithelium infected with HSV-1 17 GFP and treated with PBA (10 mM). FIG. 4F shows stereoscopic images of the eyes of C57BL6 mice infected with 5×105 PFU HSV-1 McKrae after corneal epithelial debridement and treated with ACV (5 mg/kg), PBA (50 mg/kg) or PBS mock. FIG. 4G shows the results of a plaque assay of ocular swabs to determine viral titers. FIG. 4H shows stereoscopic images of the genitalia of female BALB/C mice infected with 5×105 PFU HSV-2 333 and treated with ACV (5 mg/kg), PBA (50 mg/kg) or PBS mock. FIG. 4I shows the plaque assay results obtained from vaginal swabs to assess the viral titer.

FIGS. 5A-5I show that PBA synergizes with trifluridine (TFT) to reduce effective antiviral concentration and ocular toxicity. FIG. 5A shows fluorescent images of HCEs infected with K26 RFP were treated with PBA alone, TFT alone or PBA+TFT cocktail at shown concentrations. FIG. 5B shows the results of a plaque assay of cell lysates to assess the viral titer. FIG. 5C shows stereroscopic images of the eyes of C57BL6 mice infected with PFU HSV-1 McKrae and treated with TFT (25 μM), PBA (5 mM), PBA (5 mM)+TFT (25 μM) cocktail or DMSO control. FIG. 5D shows direct images (left) or cryo-sectioned and stained images of the mouse eyes. FIG. 5E shows the results of a plaque assay of ocular swabs to assess the viral titer. FIG. 5F shows eye surface dryness of C57BL6 mice that were dosed every day with TFT (25 μM), PBA (5 mM), PBA (5 mM)+TFT (25 μM) cocktail or DMSO control, 3 times a day for 4 weeks. FIG. 5G shows optical coherence tomography assessing corneal inflammation. FIG. 5H shows the results of a corneal sensitivity test through blink response using an esthesiometer. FIG. 5I shows the intra ocular pressure of anesthetized mice.

FIGS. 6A-6E show that synergizing Acyclovir with PBA can reduce the effective dose required to control HSV-1 encephalitis. FIG. 6A shows that BALB/C mice were infected with 1×105 PFU HSV-1 McKrae through intranasal route to induce encephalitis. FIG. 6B shows that infected mice when treated with ACV look healthier with no behavioral changes (left) compared those left untreated (right). FIG. 6C shows survival rates for infected mice that were administered ACV (50 and 10 mg/kg), PBA (100, 400 mg/kg) or a cocktail of PBA (100 mg/kg)+ACV (10 mg/kg). FIG. 6D shows weight loss or behavioral changes of studied animals. FIG. 6E shows daily disease progression and score.

FIG. 7 shows that CREB3 Like proteins are expressed differently during HSV-1 infection.

FIGS. 8A and 8B show HCEs that were transfected with either scrambled or CREB3 siRNA for a period of 24 hours. FIG. 8A shows quantification of western blot data showing decrease in CREB3 expression when CREB3 siRNA was used as opposed to scrambled RNA. FIG. 8B shows siRNA transfected HCE cells infected with 0.1 MOI HSV-1 17 GFP. At 24 hpi, flow cytometry analysis was performed to quantify extent of viral infection (more green fluorescence=more infection).

FIG. 9 shows HCEs that were transfected with either empty vector PcDNA or CREB3 overexpression plasmid for a period of 24 hours prior to infecting them with mock or 0.1 MOI HSV-1 17 GFP. At 24 hpi. cell lysates and cell culture supernatant was collected and used as inoculum to assess intracellular and extracellular virus respectively via plaque assay on Vero cells.

FIG. 10 shows salubrinal treated, HSV-1 infected HCEs form large syncytia and survive longer in cell culture.

FIG. 11 shows HCE cells that were either treated with Tunicamycin (TM), Thapsigargin (TG), Salubrinal (sal) or PBA for a period of 24 hours prior to collecting the cells for immunoblotting to check for the expression of CREB3 and CHOP in HCEs. GAPDH was used as loading control.

FIG. 12 shows mean fluorescent intensity of HSV-1 infected human skin grafts.

FIG. 13 shows trigeminal ganglia collected from HSV-1 infected mice day 28 post infection showing reactivation of virus from latency.

FIG. 14 shows in vitro antiviral efficacy testing of PBA on HSV-2 infected HeLa cells. (left) Fluorescent images of HeLa cells infected with HSV-2 GFP virus and treated with shown concentrations of PBA. (Right) Plaque assay from the cell lysates collected from the aforementioned experiment.

FIG. 15 shows synergy of PBA with various anti-HSV nucleoside analogs measured using a GFP producing HSV-1 reporter virus. HCEs were infected with HSV-1 17 GFP strain at an MOI of 0.1. All the samples were therapeutically treated 2 hpi with shown drugs either individually or in synergy with PBA. At 24 hpi, images were captured in the green channel to showcase extent of viral infection (more GFP=more HSV-1 replication and spread).

FIGS. 16A and 16B show HCEs that were infected with a 8-galactosidase producing HSV-1 virus strain TK-12 which is resistant to acyclovir (ACV). At 2 hpi, the cells were treated with DMSO, ACV or PBA for a period of 24 hours. FIG. 16A—Brightfield images were procured at 24 hpi which show syncytial structures characteristic to HSV infection FIG. 16B—Extent of virus replication was evaluated by the addition of 8-galactosidase substrate, o-nitrophenyl-8-d-galactopyranoside (ONPG) to the cells and absorption was measured using a plate reader at 405 nm.

FIG. 17 shows antiviral efficacy of PBA against bovine herpesvirus (BHV) and pseudorabies virus (PRV) at various concentrations. HCE cells were infected with 0.1 MOI of the virus and treated with shown concentrations for 24 hours. At 24 hpi, cells were lysed and the virus from the cell lysates was overlaid on Vero Cells to perform a plaque assay.

FIG. 18 shows a pathway depicting upregulation of CREB3 and down-regulation of ATF4 and CHOP during HSV infection and our proposed mechanism of action of PBA in alleviating ER stress and upregulating CHOP-mediated apoptosis in infected cells. When human corneal epithelial cells are infected with HSV-1, most UPR associated genes are silenced, except for CREB3, which then translocates to the nucleus and upregulates the transcription of pro-survival factors. On the other hand, when HSV-1 infected cells are treated with PBA, it alleviates ER stress and promotes ATF4 and CHOP translocation to the nucleus, resulting in the apoptosis of infected cells.

FIG. 19 shows that PBA ocular treatment ameliorates HSV-1 induced corneal keratitis. Mice were infected for 28 days with HSV-1 in the cornea. Only animals with keratitis were chosen and treated for 4 weeks with PBA or DMSO. FIG. 19A shows that weekly OCT assessment of the murine eyes to measure corneal thickness. FIG. 19B shows that quantitative assessment of murine corneal thickness from FIG. 19A. FIG. 19C shows IHC of murine corneas, and FIG. 19D shows the quantification of corneal thickness for different treatment groups.

FIG. 20 shows ocular surface dryness scores assessed through blind reviewers using the fluorescent images of eyes of C57BL6 mice dosed every day with TFT (25 μM), PBA (5 mM), PBA (5 mM)+TFT (25 μM) cocktail or DMSO control, 3 times a day for 4 weeks.

### DETAILED DESCRIPTION

As set forth herein, Herpes simplex virus type-1 (HSV-1) is a leading cause of infectious blindness and consequential corneal transplants in the USA with limited treatment options. The current treatment options include acyclovir and its derivatives, ganciclovir and foscarnet. All these drugs primarily act upon a single aspect of the viral lifecycle and hence work the same way. While these options are effective and have shown promise in reducing ocular HSV-1 infection, emergence of drug resistance in the recent years has caused significant distress in the infected community. Therefore, new antiviral treatments that not only act via a different mode of action but also improve the health of the infected cell by reducing ER Stress are needed and achievable, in accordance with the principles herein. 4-Phenylbutyrate (4PBA) is a known and well-studied ER chaperone that helps alleviate ER stress by increasing protein folding capacity in the ER and effectively translocating misfolded proteins for proteosomal degradation. This compound has been used to treat urea cycle disorders to help the excretion of excess nitrogen. It is an orphan drug, marketed by Ucyclyd Pharma under the trade name Buphenyl, by Swedish Orphan International (Sweden) as Ammonaps, and by Fyrlklovern Scandinavia as triButyrate. Through in vitro and in vivo studies, the disclosure herein demonstrates that PBA and its water soluble counterpart sodium phenylbutyrate (NaPBA) suppressed viral protein synthesis and therefore PBA and NaPBA as promising alternatives to current HSV antiviral therapies.

Herpes simplex virus-1 (HSV-1) causes recurrent infection of the eyes resulting in virus shedding (symptomatic or asymptomatic), inflammation and scarring of the cornea. It is among the most frequent causes of corneal opacification and corneal transplants in the modern world. Nucleoside analogs (such as acyclovir (ACV)) are effective in controlling symptoms, but are susceptible to resistance or show significant toxicity after long-term use.

In accordance with the principles herein, HSV-1, HSV-2 and other human herpes viruses and animal herpesviruses can be treated using an alternate mode of treatment. PBA and the disclosed compositions act on an alternate mode of action to ACV. Furhtermore, PBA and the disclosed composition may be administered in addition to ACV, which results inet a combined drug competence.

HSV and Endoplasmic Reticulum (ER) Stress

The unfolded protein response (UPR) is an important cellular pathway that is initiated as a result of endoplasmic reticulum (ER) stress. ER stress is caused as a result of protein accumulation due to excessive misfolded, unfolded proteins in the ER. UPR is the cell's natural response to such events where (a) new protein synthesis is stalled (b) protein folding chaperones are upregulated to ensure timely protein folding and (c) misfolded proteins are directed towards degradation. Multiple reports in the past have shed light on the cell's UPR pathway to ER stress and have shown that these three legs of UPR when functioning properly can lead to either the alleviation of stress and hence survival of the cell or stimulation of apoptosis pathways that lead to cell death. These checks and balances not only ensure healthy maintenance of the cell but protect a colony of cells from external infections.

However, studies with HSV have shown that this virus effectively disarms the UPR while extensively producing, folding and secreting viral proteins from the ER without triggering cell death pathways. This robust modulation of UPR by the virus has intrigued the interest of herpes virologists to uncover underlying mechanisms, which can be exploited to control virus replication. Multiple studies in the past have used small molecules such as Thapsigargin (TG), Brefeldin-A (BA) and Tunicamycin (TM) to disrupt viral replication via the inhibition of protein translation. However, these drugs at effective antiviral concentrations also cause acute ER stress, which could lead to other systemic disorders in neighboring non-infected cells.

In accordance with the principles herein, modulation of ER-resident pro-survival transcription factor cAMP response element-binding protein-3 (CREB3) by HSV-1 is set forth. HSV-1 downregulates CCAAT-enhancer-binding protein homologous protein (CHOP), an ER-stress dependent apoptosis regulator through the modulation of CREB3. Without wishing to be bound by any particular theory, with the help of a chemical chaperone 4-Phenylbutyrate (PBA), the alleviation of ER stress can curb HSV infection in the eye, genitals and also protect from encephalitic death. Additionally, ocular toxicity profile of a known topical antiviral and how its toxic effects can be alleviated using PBA in synergy is set forth.

Upregulation of CREB3 in HSV-1 Infection

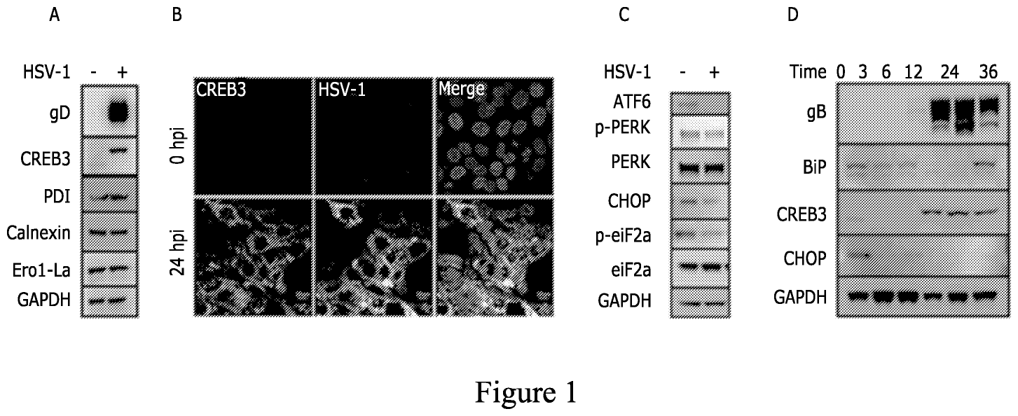

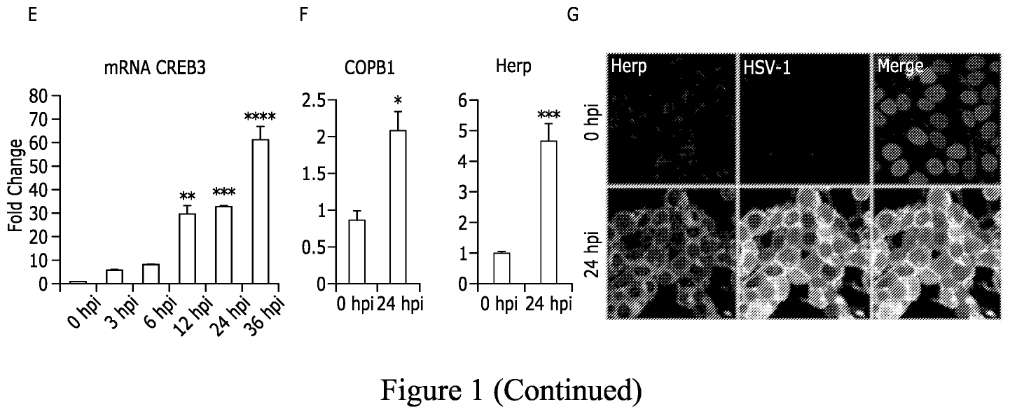

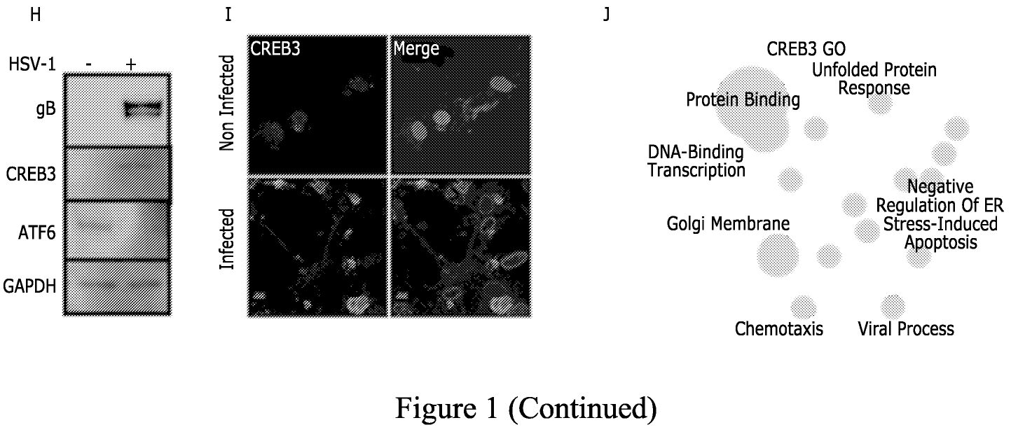

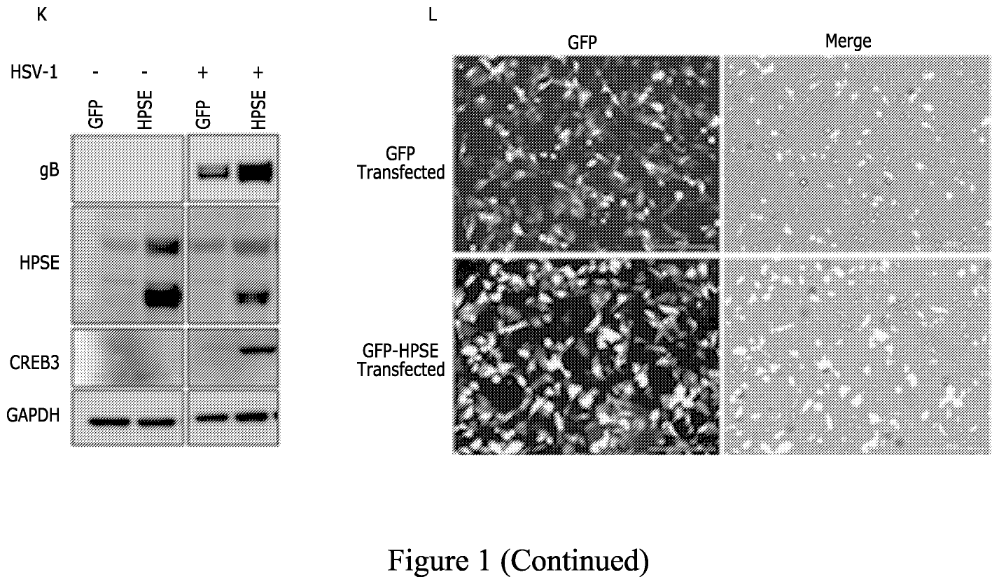

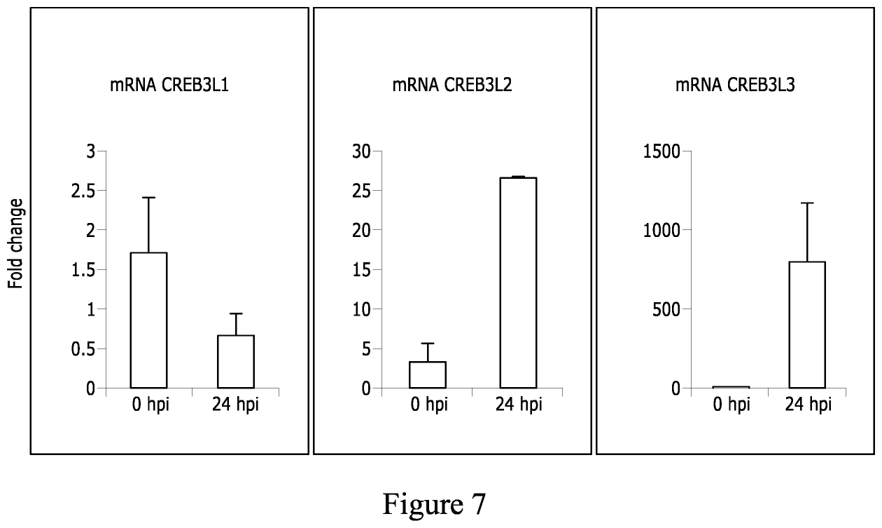

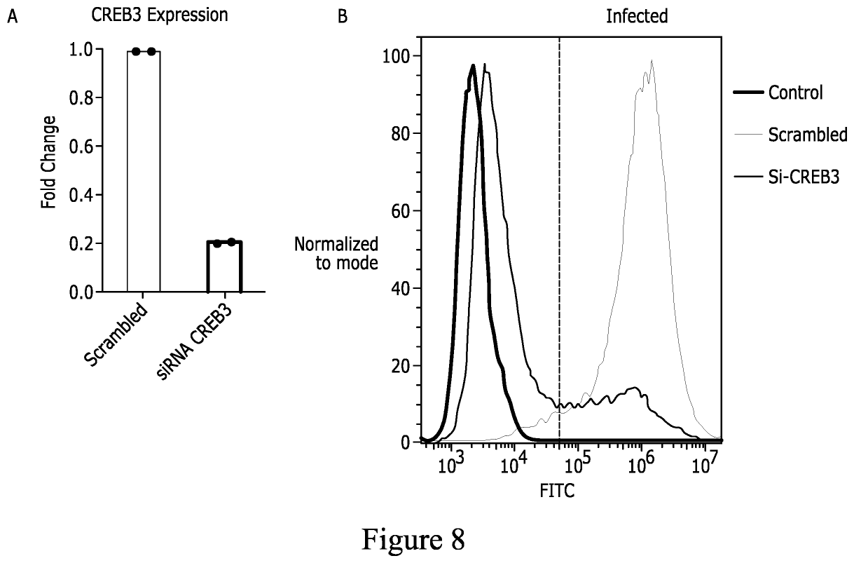

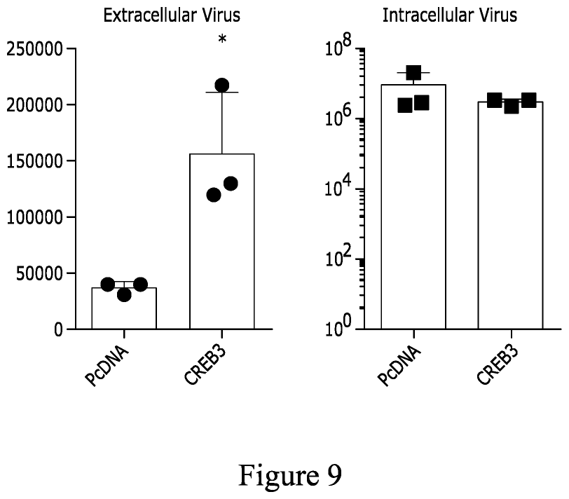

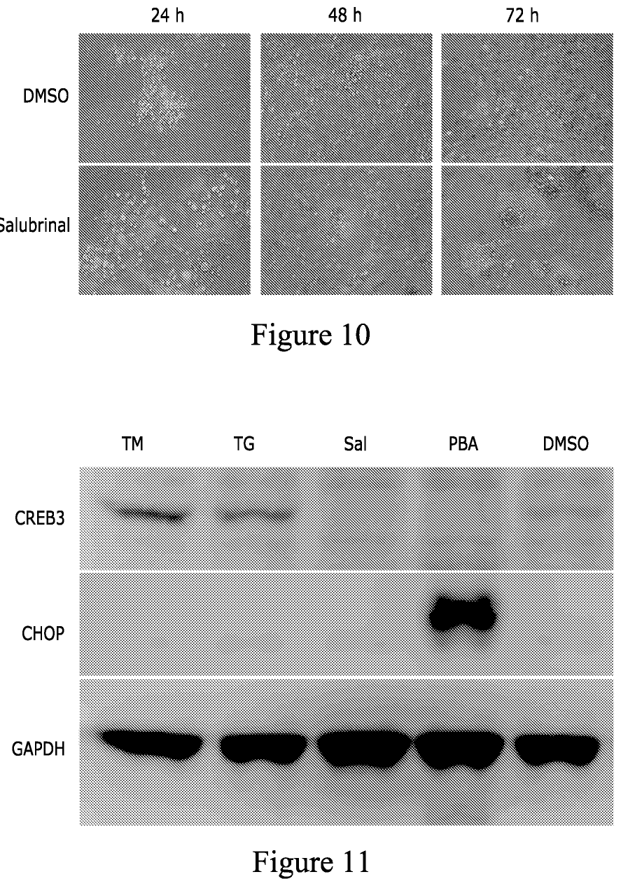

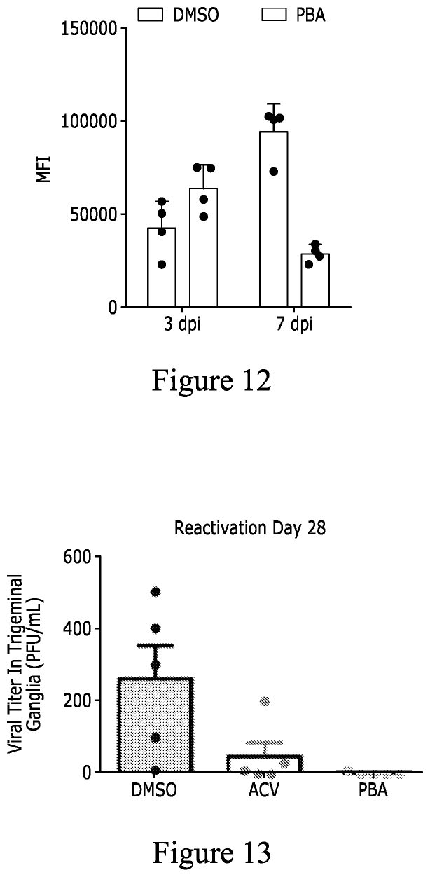

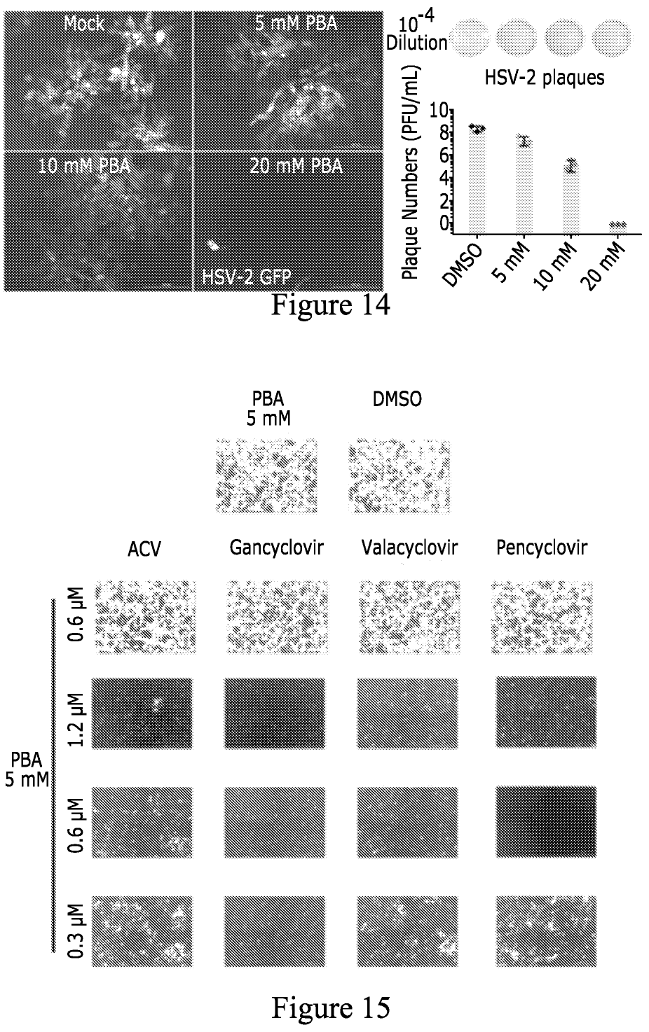

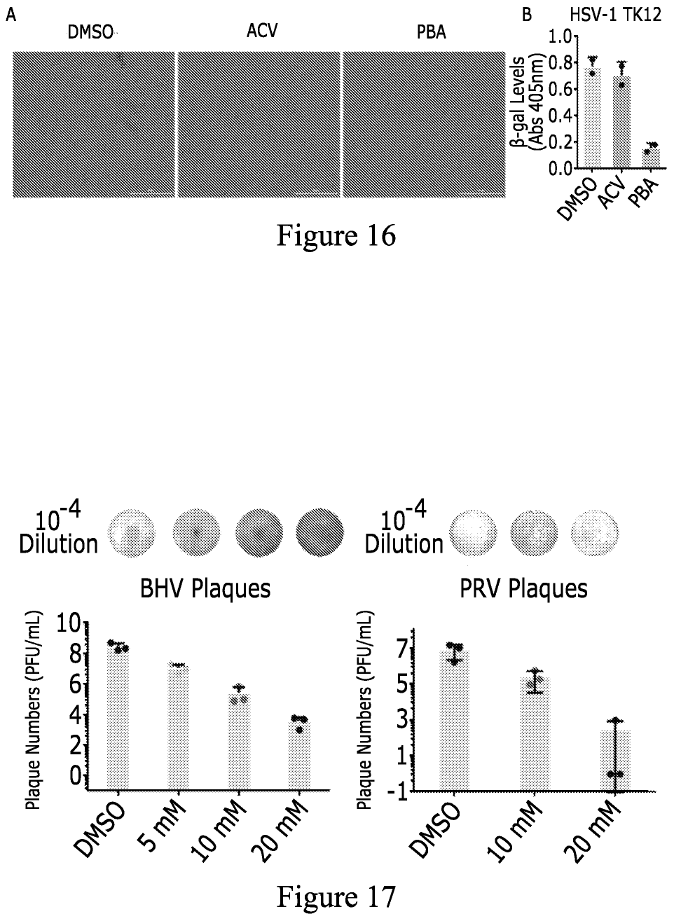

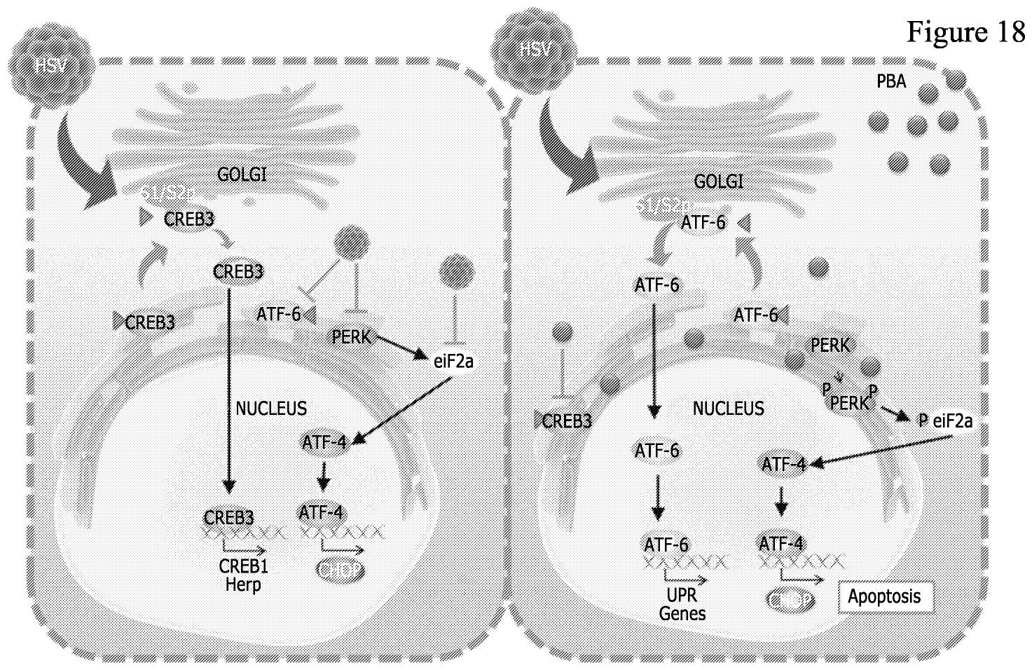

To understand the behavior of UPR during HSV-1 infection, an unbiased proteomic analysis of all the ER resident regulators using western blotting technique on human corneal epithelial cell line (HCEs) was conducted. Of all the proteins surveyed, only CREB3 was upregulated during infection, while all the other master regulators such as activating transcription factor (ATF)-6, phosphorylated protein kinase-R like ER-kinase (PERK), phosphorylated eukaryotic translation initiation factor (eIF) 2A, CHOP calnexin etc., were either downregulated or remained the same (FIGS. 1A, 1B and 1C). In an infection time series, CREB3 transcription and translation is upregulated (FIG. 1D, 1E) approximately 12 hours post infection (hpi) in addition to the upregulation of its downstream components (FIG. 1F, 1G) such as Coatomer subunit beta (COPB1) and Homocysteine-induced endoplasmic reticulum protein (herp) at 24 hpi. Most importantly, CHOP, the ER-stress dependent apoptosis factor was inhibited upon viral infection. These results correlated with results in primary human corneal epithelial cells (pHCEs) that were freshly isolated from human donors (FIG. 1H, 1I). Given that the gene ontology (GO) of CREB3 is associated with multiple cellular processes including the negative regulation of ER stress-induced apoptosis, the effect of heparanase (HPSE), another virally modulated pro-survival factor, on CREB3 expression was analyzed. Interestingly, while there was no change in CREB3 expression in mock-infected cells, there was a >10 fold higher expression in HPSE overexpressing infected cells (FIG. 1K, 1L). Furthermore, a differential expression in CREB3 like proteins CREB3L1, CREB3L2, and CREB3L3 was found (FIG. 7).

ER stress is one of the main stress pathways that the virus uses to regulate its replication inside a host cell. Without wishing to be bound by any particular theory, this is believed to stem from the virus controlling the three important ER membrane proteins to its own advantage, thereby regulating the three main branches of UPR. Again without wishing to be bound by any particular theory, three ER transmembrane proteins, Inositol Requiring 1 (IRE1), PERK, and ATF6 are believed to be responsible for the activation of UPR. An ER chaperone, immunoglobin binding protein (BiP) binds to the luminal domains of these master regulators during unstressed conditions, keeping them inactive. Upon ER stress, BiP dissociates from these sensors resulting to their activation. Activated IRE1 splices a ubiquitously present cytosolic protein X-box protein 1 (XBP-1) resulting in its translocation to the nucleus. Activated PERK phosphorylates eiF2α resulting in the deactivation of protein translation. ATF6, upon activation proteolytically cleaves from its 90 kDa transmembrane form to 50 kDa free form which translocates to the nucleus. Downstream effectors of IRE1 and ATF6 upregulate UPR genes via the ER stress element (ERSE) and UPR element (UPRE) in the nucleus. This results in the production of chaperone proteins and ER associated degradation (ERAD) proteins which help in the proper folding of unfolded proteins in the ER or retrotranslocation and degradation of misfolded proteins. In the case where either of these strategies fail, chronic ER stress ensues resulting in a final and more absolute measure to initiate apoptotic cell death via the activation of ATF4 and CHOP.

Another major component of the ER and UPR is CREB3/LZIP/LUMAN, a member of the leucine zipper family of DNA binding proteins, was identified for its interaction with human cell factor complex-1 (HCFC1) during ER stress. During ER stress, CREB3 translocates to the Golgi where it underdoes regulated intramembrane proteolysis (RIP) through S1P and S2P protease based activation. During HSV-1 infection, VP16, a viral homolog of CREB3 has been shown to bind several host proteins including HCFC1 to initiate viral transcription. CREB3's interaction with HCFC1 was proposed to retain HCFC1 in the ER, preventing it from facilitating transcription activated by HSV-1 VP16. However most of these models were proposed in neuronal cell lines where the over-expression of CREB3 would sequester HCFC1 in the ER, ensuring HSV-1 latency. However, in studies on both primary and HCE cell lines, it was found that CREB3 overexpression indeed results in the increase in viral egress from the cell and CREB3 knockout inhibits viral infection. This result is multifold, given that the studies herein show CREB3's pro-survival functionality is mediated through the modulation of CHOP, an ER-stress inducible apoptotic factor. Through RNA interference, it was shown that CREB3 inhibition is associated with increase in ATF4 and CHOP. All these results point to the fact that CREB3 expression, like HPSE, is modulated by HSV-1 for its own benefit whether to export virus particles to the cell surface or to inhibit apoptotic factors during lytic viral replication.

In accordance with the principles herein, during the study of the UPR, it was found that inhibition of CREB3 during HSV-1 infection resulted in significant reduction of viral replication. Currently there are no known small molecule inhibitors of CREB3. Hence previously tested ER modulators such as TM, TG, BA, salubrinal, and PBA were then studied for their effect on CREB3. It was found that ER-stress inducers such as TM, TG and BA inhibited viral replication, but also elevated the level of CREB3 in the cell. On the other hand, salubrinal, a specific inhibitor of elF2a phosphatase enzymes, showed increased survival of the cell, increased viral replication, decreased CHOP expression and no change in CREB3 levels. Only PBA, a chemical chaperone, was able to potently inhibit viral replication, decreased CREB3 expression followed by increased CHOP in the nucleus. It is important to note that PBA has previously been implicated in the abrogation of African swine fever virus and bovine herpesvirus through the inhibition of hypoacetylation status of histone H3K9/K14 and activation of inflammation related signaling including Erk1/2 and p38MAPK respectively. Also, other studies have shown the beneficial effects of administering PBA during Salmonella Typhimurium infection. However, the correlation between the use of PBA on the inhibition of HSV in target cell lines was unknown until now. In vitro studies on bovine herpes virus and pseudorabies viruses (FIG. 17) showed that PBA has potent antiviral activity against both of them, suggesting a broad antiviral activity against all alpha-herpesviruses.

Given the promise of PBA, its activity in primary human corneas, primary HCEs, human ex vivo skin grafts and in murine models of ocular, genital and encephalitic infection were studied. In all the cases, PBA showed excellent antiviral activity comparable to ACV, a clinically used anti-herpetic drug. Interestingly, PBA has the ability to synergize with known anti-herpetic drugs.

A phase II clinical trial, based on an initial publication, studied the anti-cancer efficacy of combining valganciclovir with PBA in patients with relapsed or refractory Epstein-Barr virus-positive cancer. Topical drugs such as TFT, are required to be dosed up to 10 times every day, although novel sustained release formulations can reduce the dosing frequency. Prolonged use of TFT has been associated with corneal toxicity including inflammation, dryness and pain. Systemic drugs such as acyclovir, although very effective, can cause renal toxicity including acute kidney injury when administered intravenously. Synergizing these anti-herpetic drugs with PBA, can indeed reduce the serious toxic effects associated with them.

PBA is a well-known and well-studied ER chaperone that helps alleviate ER stress by increasing protein folding capacity in the ER and effectively translocating misfolded proteins for proteosomal degradation. This compound is used to treat urea cycle disorders where it helps the excretion of excess nitrogen. Attributing to these qualities, therapies which synergize with PBA would provide additional renal protection when higher dosages of antiviral drugs need to be administered systemically Given that PBA has shown the ability to cross the blood brain barrier, the applications for its use will have greater implications in future antiviral compounds and compositions constructed in accordance with the principles herein.

Compositions, Pharmaceutical Formulations, and Modes of Administration

Provided herein are compositions comprising: 4-phenylbutyrate (PBA) or a pharmaceutically acceptable salt thereof in an effective concentration as a therapeutic agent against viral infections.

In some cases, the PBA is present as a salt. In some cases, the PBA is sodium 4-phenylbutanoate.

In some cases, the concentration of the PBA or salt thereof in the composition ranges from about 1 mM to about 200 mM, or has a cumulative concentration ranging from about 10 mg/kg to about 600 mg/kg.

In some cases, the concentration of the PBA or salt thereof in the composition ranges from about 1 mM to about 200 mM. In some cases, In some cases, the concentration of the PBA or salt thereof in the composition ranges from about 1 mM to about mM, from about 1 mM to about 10 mM, from about 1 mM to about 20 mM, from about 1 mM to about 30 mM, from about 1 mM to about 40 mM, from about 1 mM to about 50 mM, from about 1 mM to about 60 mM, from about 1 mM to about 70 mM, from about 1 mM to about 80 mM, from about 1 mM to about 90 mM, from about 1 mM to about 100 mM, from about 1 mM to about 110 mM, from about 1 mM to about 120 mM, from about 1 mM to about 130 mM, from about 1 mM to about 140 mM, from about 1 mM to about 150 mM, from about 1 mM to about 160 mM, from about 1 mM to about 170 mM, from about 1 mM to about 180 mM, from about 1 mM to about 190 mM, or from about 1 mM to about 200 mM. In some cases, In some cases, the concentration of the PBA or salt thereof in the composition ranges from about 1 mM to about 50 mM, from about 50 mM to about 100 mM, from about 100 mM to about 150 mM, or from about 150 mM to about 200 mM. In some cases, the concentration of the PBA or salt thereof in the composition ranges from about 1 mM to about 200 mM, about 10 mM to about 200 mM, about 20 mM to about 200 mM, about 30 mM to about 200 mM, about 40 mM to about 200 mM, about 50 mM to about 200 mM, about 60 mM to about 200 mM, about 70 mM to about 200 mM, about 80 mM to about 200 mM, about mM to about 200 mM, about 100 mM to about 200 mM, about 110 mM to about 200 mM, about 120 mM to about 200 mM, about 130 mM to about 200 mM, about 140 mM to about 200 mM, about 150 mM to about 200 mM, about 160 mM to about 200 mM, about 170 mM to about 200 mM, about 180 mM to about 200 mM, or about 190 mM to about 200 mM. In some cases, the concentration of the PBA or salt thereof in the composition is about 1 mM, about 10 mM, about 20 mM, about 30 mM, about 40 mM, about 50 mM, about 60 mM, about mM, about 80 mM, about 90 mM, about 100 mM, about 110 mM, about 120 mM, about 130 mM, about 140 mM, about 150 mM, about 160 mM, about 170 mM, about 180 mM, about 190 mM, or about 200 mM. In some cases, the concentration of the PBA or salt thereof in the composition is about 20 mM.

In some cases, the composition of the PBA or salt thereof in the composition has a cumulative concentration ranging from about 10 mg/kg to about 600 mg/kg. In some cases, the concentration of the PBA or salt thereof ranges from about 10 mg/kg to about 100 mg/kg, from about 10 mg/kg to about 200 mg/kg, about 10 mg/kg to about 300 mg/kg, about 10 mg/kg to about 400 mg/kg, about 10 mg/kg to about 500 mg/kg, or about 10 mg/kg to about 600 mg/kg. In some cases, the concentration of the PBA or salt thereof ranges from about 100 mg/kg to about 600 mg/kg, about 200 mg/kg to about 600 mg/kg, about 300 mg/kg to about 600 mg/kg, about 400 mg/kg to about 600 mg/kg, or about 500 mg/kg to about 600 mg/kg. In some cases, the concentration of the PBA or salt thereof is about 100 mg/kg, about 200 mg/kg, about 300 mg/kg, about 400 mg/kg, about 500 mg/kg, or about 600 mg/kg. In some cases, the concentration of the PBA or salt thereof is about 100 mg/kg.

As used herein, “subject” refers to any mammal, including humans, domestic and farm animals, and zoo, sports, or pet animals, such as dogs, horses, cats, sheep, pigs, cows, etc. The preferred mammal herein is a human, including adults, children, and the elderly. Preferred sports animals are horses and dogs. Preferred farm animals are cows, pigs, horses, goats and sheep. Preferred pet animals are dogs and cats.

As used herein, a “therapeutically effective amount” in reference to the disclosed HPAC compositions or therapeutic agents refers to the amount sufficient to induce a desired biological, pharmaceutical, or therapeutic result. That result can be treating, reducing or preventing of the signs, symptoms, or causes of a disease or disorder or condition, or any other desired alteration of a biological system. In regards to antiviral agents, a therapeutically effective amount reduces or prevents virus replication, reduces viral load, reduces or prevents viral spread, e.g. inhibiting syncytia formation, and/or reduces or prevents viral entry into a cell.

As used herein, the terms “treating” and “treatment” refer to both therapeutic treatment and prophylactic or preventative measures.

In exemplary aspects, the compositions disclosed herein comprise PBA or a pharmaceutically acceptable salt thereof and a drug delivery agent. In some cases, the drug delivery agent is a pharmaceutically acceptable carrier, diluent, or excipient. In exemplary aspects, the compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopedia for use in animals, including humans. In some cases, the drug delivery agent is selected from a polymeric agent, activated carbon agent, metal agent, metal oxide agent, liposomal agent, peptide/protein agent, sugar agent, and combinations thereof. In some cases, the polymeric agent is a Eudragit® polymer.

The compositions in various aspects comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

In exemplary aspects, the compositions comprise formulation materials that are nontoxic to recipients at the dosages and concentrations employed. In specific embodiments, pharmaceutical compositions comprising PBA and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In exemplary aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the compositions comprise formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as bcnzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbatc, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

The compositions in various instances are formulated to achieve a physiologically compatible pH. In exemplary embodiments, the pH of the compositions is for example between about 4 or about 5 and about 8.0 or about 4.5 and about 7.5 or about 5.0 to about 7.5. In exemplary embodiments, the pH of the composition is between 5.5 and 7.5.

The compositions may be administered to a subject via parenteral, nasal, oral, pulmonary, topical, ophthalmic, vaginal, or rectal administration. The following discussion on routes of administration is merely provided to illustrate exemplary embodiments and should not be construed as limiting the scope in any way.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. PBA in various instances is administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-6-aminopropionates, and 2-alkyl-imidazoline quaternary ammonium salts, and (e) mixtures thereof.

Preservatives and buffers can be used. In order to minimize or eliminate irritation at the site of injection, such compositions can contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the present disclosure. The requirements for effective pharmaceutical carriers for injectable compositions are well-known to those of ordinary skill in the art (see, e.g., Pharmaceutics and Pharmacy Practice, J. B. Lippincott Company, Philadelphia, PA, Banker and Chalmers, eds., pages 238-250 (1982), and ASHP Handbook on Injectable Drugs, Toissel, 4th ed., pages 622-630 (1986)).

Formulations suitable for oral administration in some aspects comprise (a) liquid solutions, such as an effective amount of PBA dissolved in diluents, such as water, saline, or orange juice; (b) capsules, sachets, tablets, lozenges, and troches, each containing a predetermined amount of HPAC, as solids or granules; (c) powders; (d) suspensions in an appropriate liquid; and (e) suitable emulsions. Liquid formulations in some aspects include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise HPAC in a flavor, usually sucrose and acacia or tragacanth, as well as pastilles comprising HPAC in an inert base, such as gelatin and glycerin, or sucrose and acacia, emulsions, gels, and the like containing, in addition to, such excipients as are known in the art.

In some cases, the compositions comprise PBA or a pharmaceutically acceptable salt thereof and a drug delivery agent as a nanoformulation (i.e., a formulation comprising particles of the PBA in nanometer size), microformulation (i.e., a formulation comprising particles of the PBA in micrometer size), or macroformulation (i.e., a formulation comprising particles of the PBA in larger than micrometer size).

Topical Delivery

In some embodiments, a composition is formulated, for example, as a topical (e.g., dermal) formulation. In some embodiments, a composition is formulated, for example, for topical administration to a mammal. A topical formulation may include, for example, a formulation such as a gel formulation, a cream formulation, a lotion formulation, a paste formulation, an ointment formulation, an oil formulation, and a foam formulation. The composition further may include, for example, an absorption emollient.

Additional examples of a composition can optionally be formulated to be delivered to the mucosum, or by inhalation, respiration, intranasal, oral, buccal, or sublingual.

Salts may be added. Non-limiting examples of salts include acetate, benzoate, besylate, bitartate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulphate, mucate, napsylate, nitrate, pamoate (embonate, phosphate, diphosphate, salicylate and disalicylate, stearate, succinate, sulphate, tartrate, tosylate, triethiodide, valerate, aluminium, benzathine, calcium, ethylene diamine, lysine, magnesium, megluminie, potassium, procaine, sodium, tromethyamine or zinc.

Topical (skin, e.g., face) formulations can include, for example, a liquid or cream with or without moisturizer. Components of a liquid or cream with moisturizer (moisturizing formulation) can be: Colloidal oatmeal, niacinamide, creamides, phsospholipids, triglycerides, fats or fatty acids, free fatty alcohols, waxes (esters, diesters, triesters, etc.), hydroxyacid diesters, squalene, sterol esters, cholesterol, lactones, etc. In addition, the topical formulations can be incorporated as creams, gels, or foams to serve as topical treatment for viral infection or for rectal or vaginal application (e.g. to mucosal surfaces)

Suitable carrier materials for use in sustained release delivery devices of the disclosure include any carrier or vehicle commonly used as a base for creams, lotions, gels, emulsions, lotions or paints for topical administration. Examples include emulsifying agents, inert carriers including hydrocarbon bases, emulsifying bases, non-toxic solvents or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol.

Suitable carriers include: pluronic gels, polaxamer gels, hydrogels containing cellulose derivatives, including hydroxyethyl cellulose, hydroxymethyl cellulose, carboxymethyl cellulose, hydroxypropylmethyl cellulose and mixtures thereof, and hydrogels containing polyacrylic acid (Carbopols). Suitable carriers also include creams/ointments used for topical pharmaceutical preparations, e.g., creams based on cetomacrogol emulsifying ointment. The above carriers may include or exclude, for example, alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA.

In some cases, the composition comprises a mucosal penetrating agent. In some cases, the mucosal penetrating agent comprises poloxamer 407.

Ophthalmic Administration

In some embodiments, the compositions described herein are formulated for ophthalmic administration, including, for example, intravitreal or intraocular administration. Typically, compositions for administration comprise sterile isotonic aqueous buffer. Where necessary, the compositions can also include a solubilizing agent. Also, the agents can be delivered with a suitable vehicle or delivery device as known in the art. Combination therapies described herein can be co-delivered in a single delivery vehicle or delivery device. Compositions for administration can optionally include a local anesthetic such as lignocaine to lessen pain at the site of the injection.

Compositions for ophthalmic administration can be formulated without limitation as an intra-ocular injection, intra-vitreal injection, topical ophthalmic drops, sub-conjunctival injection, sub-Tenon's injection, trans-scleral formulations, and the like. In some cases, the compositions may be formulated as micronized suspensions in isotonic, pH adjusted sterile saline, or, specifically, as solutions in isotonic, pH adjusted sterile saline, either with or without a preservative such as benzalkonium chloride. Alternatively, for ophthalmic uses, the compositions may be formulated in an ointment such as petrolatum.

In some cases, the composition for ophthalmic administration comprises an ophthalmic excipient. In some cases, the ophthalmic excipient is selected from Cyclodextrins, Carbopol or carbomer or acrylic acid polymers, Poloxamers, Xyloglucan, Methylcellulose, Hydroxypropyl Methylcellulose, Ethyl (Hydroxyethyl) Cellulose, Pseudolatexes, Cellulose Acetate Phthalate, Gellan Gum, Alginate, Carrageenans, Hyaluronic Acid, Sodium acetate, Edetate disodium, Hypromellose, Acetic acid, Alcohol, Alginic acid, Amerchol-cab, Antipyrine, Benzalkonium chloride, Benzododecinium bromide, Boric acid, Caffeine, Calcium chloride, Carbomer 1342, Carbomer 934P, Carbomer 940, Carbomer homopolymer type B (allyl pentaerythritol cross-linked), Carboxymethylcellulose sodium, Castor oil, Cetyl alcohol, Chlorobutanol, Citric acid, Citric acid monohydrate, Creatinine, Divinylbenzene styrene copolymer, Ethylene vinyl acetate copolymer, Gellan gum (low acyl), Glycerin, Glyceryl stearate, Hypromelloses, Lanolin, Lauralkonium chloride, Lauroyl sarcosine, Magnesium chloride, Methylparaben, Mineral oil, Nonoxynol-9, Octoxynol-40, Petrolatum, Phenylethyl alcohol, Phenylmercuric acetate, Phenylmercuric nitrate, Polidronium chloride, Poloxamer 188 or 407, Polycarbophil, Polyethylene glycol 400 or 8000, Polyoxyl 35 castor oil, Polyoxyl 40 hydrogenated castor oil, Polyoxyl 40 stearate, Polypropylene glycol, Polysorbate 20, Polyvinyl alcohol, Potassium chloride, Potassium sorbate, Povidone K29/32, Povidone K30, Povidone K90, Povidones, Propylene glycol, Propylparaben, Soda ash, Sodium acetate, Sodium bisulfate, Sodium borate, Sodium borate decahydrate, Sodium carbonate, Sodium chloride, Sodium citrate, Sodium metabisulfite, Sodium nitrate, Sodium sulfate, Sodium sulfite, Sodium thiosulfate, Sorbic acid, Sorbitol, Stabilized oxychloro complex, Sulfuric acid, Thimerosal, Titanium dioxide, Tocophersolan, Trisodium citrate dehydrate, Tromethamine, Tyloxapol, Xanthan gum, Zinc chloride, and combinations thereof.

Therapeutic Agents

The disclosure provides for compositions comprising PAB or a pharmaceutically acceptable salt thereof and one or more additional therapeutic agents. The term “therapeutic agent” refers to any agent or compound known in the art that elicits a therapeutic effect or treats or reduces or prevents the onset, duration or effect of a symptom of a disease, condition or disorder and/or the onset, duration or effect of the disease, condition or disorder itself.

The therapeutic agents include antiviral agents, antibacterial agents, anticancer and cytotoxic agents, analgesics, anti-hypertension drugs, anti-allergenic (anti-histamine), an anti-seizure compound, non-steroidal anti-inflammatory drugs, an antibiotic, growth hormone, parathyroid hormone, insulin, interferons, chemotherapeutic agents, glucagon like peptides (e.g., GLP-1, exenatide), polynucleotides including DNA and RNA such as siRNA and shRNA, plasmids and vectors, DNA based compounds that can target viral/non-viral targets, peptides based on compounds that can target viral/non-viral targets, parathyroid hormones, growth hormones (e.g., IFG and other growth factors), immune suppression agents, and anti-parasitic agents such as various anti-malarial agents. In addition, therapeutic agents include natural enzymes, proteins derived from natural sources, recombinant proteins, natural peptides, synthetic peptides, cyclic peptides, antibodies, receptor agonists, cytotoxic agents, immunoglobins, beta-adrenergic blocking agents, calcium channel blockers, coronary vasodilators, cardiac glycosides, antiarrhythmics, cardiac sympathomemetics, angiotensin converting enzyme (ACE) inhibitors, diuretics, inotropes, cholesterol and triglyceride reducers, bile acid sequestrants, fibrates, 3-hydroxy-3-methylgluteryl (HMG)-CoA reductase inhibitors, niacin derivatives, antiadrenergic agents, alpha-adrenergic blocking agents, centrally acting antiadrenergic agents, vasodilators, potassium-sparing agents, thiazides and related agents, angiotensin II receptor antagonists, peripheral vasodilators, antiandrogens, estrogens, antibiotics, retinoids, insulins and analogs, alpha-glucosidase inhibitors, biguanides, meglitinides, sulfonylureas, thizaolidinediones, androgens, progestogens, bone metabolism regulators, anterior pituitary hormones, hypothalamic hormones, posterior pituitary hormones, gonadotropins, gonadotropin-releasing hormone antagonists, ovulation stimulants, selective estrogen receptor modulators, antithyroid agents, thyroid hormones, bulk forming agents, laxatives, antiperistaltics, flora modifiers, intestinal adsorbents, intestinal anti-infectives, antianorexic, anticachexic, antibulimics, appetite suppressants, antiobesity agents, antacids, upper gastrointestinal tract agents, anticholinergic agents, aminosalicylic acid derivatives, biological response modifiers, corticosteroids, antispasmodics, 5-HT₄ partial agonists, antihistamines, cannabinoids, dopamine antagonists, serotonin antagonists, cytoprotectives, histamine H2-receptor antagonists, mucosal protective agent, proton pump inhibitors, H. pylori eradication therapy, erythropoieses stimulants, hematopoietic agents, anemia agents, heparins, antifibrinolytics, hemostatics, blood coagulation factors, adenosine diphosphate inhibitors, glycoprotein receptor inhibitors, fibrinogen-platelet binding inhibitors, thromboxane-A2 inhibitors, plasminogen activators, antithrombotic agents, glucocorticoids, mineralcorticoids, corticosteroids, selective immunosuppressive agents, antifungals, drugs involved in prophylactic therapy, AIDS-associated infections, cytomegalovirus, non-nucleoside reverse transcriptase inhibitors, nucleoside analog reverse transcriptase inhibitors, protease inhibitors, anemia, Kaposi's sarcoma, aminoglycosides, carbapenems, cephalosporins, glycopeptides, lincosamides, macrolies, oxazolidinones, penicillins, streptogramins, sulfonamides, trimethoprim and derivatives, tetracyclines, anthelmintics, amebicies, biguanides, cinchona alkaloids, folic acid antagonists, quinoline derivatives, Pneumocystis carinii therapy, hydrazides, imidazoles, triazoles, nitroimidzaoles, cyclic amines, neuraminidase inhibitors, nucleosides, phosphate binders, cholinesterase inhibitors, adjunctive therapy, barbiturates and derivatives, benzodiazepines, gamma aminobutyric acid derivatives, hydantoin derivatives, iminostilbene derivatives, succinimide derivatives, anticonvulsants, ergot alkaloids, antimigrane preparations, biological response modifiers, carbamic acid eaters, tricyclic derivatives, depolarizing agents, nondepolarizing agents, neuromuscular paralytic agents, CNS stimulants, dopaminergic reagents, monoamine oxidase inhibitors, COMT inhibitors, alkyl sulphonates, ethylenimines, imidazotetrazines, nitrogen mustard analogs, nitrosoureas, platinum-containing compounds, antimetabolites, purine analogs, pyrimidine analogs, urea derivatives, antracyclines, actinomycinds, camptothecin derivatives, epipodophyllotoxins, taxanes, vinca alkaloids and analogs, antiandrogens, antiestrogens, nonsteroidal aromatase inhibitors, protein kinase inhibitor antineoplastics, azaspirodecanedione derivatives, anxiolytics, stimulants, monoamine reuptake inhibitors, selective serotonin reuptake inhibitors, antidepressants, Benzisoxazole derivatives, butyrophenone derivatives, dibenzodiazepine derivatives, dibenzothiazepine derivatives, diphenylbutylpiperidine derivatives, phenothiazines, thienobenzodiazepine derivatives, thioxanthene derivatives, allergenic extracts, nonsteroidal agents, leukotriene receptor antagonists, xanthines, endothelin receptor antagonist, prostaglandins, lung surfactants, mucolytics, antimitotics, uricosurics, xanthine oxidase inhibitors, phosphodiesterase inhibitors, metheamine salts, nitrofuran derivatives, quinolones, smooth muscle relaxants, parasympathomimetic agents, halogenated hydrocarbons, esters of amino benzoic acid, amides (e.g. lidocaine, articaine hydrochloride, bupivacaine hydrochloride), antipyretics, hynotics and sedatives, cyclopyrrolones, pyrazolopyrimidines, nonsteroidal anti-inflammatory drugs, opioids, para-aminophenol derivatives, alcohol dehydrogenase inhibitor, heparin antagonists, adsorbents, emetics, opoid antagonists, cholinesterase reactivators, nicotine replacement therapy, antitussives, antiulcer agents and acid suppressants, gastrointestinal drugs, vitamin A analogs and antagonists, vitamin B analogs and antagonists, vitamin C analogs and antagonists, vitamin D analogs and antagonists, vitamin E analogs and antagonists, vitamin K analogs and antagonists or combinations thereof.

The therapeutic agent can be an analgesic or a pain relieving agent. Analgesics include ibuprofen, diphenhydramine, acetaminophen, magnesium sailicylate, aspirin, meprobamate, ziconotide, butalbitalor, codeine, hydrocodone, dihydrocodone, oxycodone, naloxone, pentazocine, fentanyl, morphine, hydromorphone, buprenorphine, methadone, meperidine, buprenorphine, oxymorphone, tramadol, nalbuphine, propoxyphene, tapentadol, alfentanil, sufentanil, remifentanil and combinations thereof. Analgesics also include antimigraine agents such as sumatriptan, ergotamine, methysergide maleate, frovatriptan, almoatriptan, ergotamine, rizatraiptan, naproxen, napatripan, eletriptan, zolmitriptan, lasmiditan, dichloralphenazone, isometreptene mucate or combinations thereof. Analgesics also include CGRP inhibitors such as erenunumab (Aimovig), fremanezumab (Ajovy), galcanezumab (Emgality) and epitinezumab. Analgesics include Cox-2 inhibitors such as celecoxib, valdecoxib, and rofecoxib. In addition, analgesics include nonsteroidal anti-inflammatory drugs such as ibuprofen, naproxen, ketoprofen, tometin, etodolac, flurbiprofen, diclofenac, misoprostol, piroxicam, fenoprofen, indomethacin, sulndac, nabumetone, ketorolac, famotidine, mefenamic acid, diflunisal, meloxicam. Analgesics include salicylates such as aspirin, salsalate, magnesium salicylate, choline salicylate, difunisal, and tricosal or combinations thereof.

The therapeutic agent can be an anti-allergenic or an anti-histamine, such as cetirizine, desloratadine, ebastine/carebastine, fexofenadine, lovocetirizine, loratadine, mizolastine, rupatadine, Bromp, heniramine, chlorpheniramine, clemastine, diphenhydramine, Ketotifen, naphazoline, pheniramine, azelastine, azelastine, pseudoephedrine, epastine and olopatadine, antihistamine drugs, such as acrivastine, astemizole, cinnarizine, cyproheptadine, dimenhydrinate, flunarizine, meclozine, oxatomide, terfenadine, and triprolidine or combinations thereof.

The therapeutic agent can be an antidepressant, such as amoxapine, ciclazindol, maprotiline, mianserin, nortriptyline, trazodone, trimipramine maleate, acetohexamide, chlorpropamide, glibenclamide, gliclazide, glipizide, tolazamide, and tolbutamide or combinations thereof.

The therapeutic agent can be an antidiabetic agent, such as acarbose, miglitol, pramlintide, alogliptan, linagliptan, saxagliptin, sitagliptin, albiglutide, dulaglutide, exenatide, liraglutide, lixisenatide, insulin, nateglinide, repaglinide, metformin, canagliflozin, dapagliflozin, empagliflozin, chlorpropamide, glimepiride, glipizide, glyburide, tolazamide, tolbutamide, rosiglitazone, and pioglitazone; alpha-glucosidase inhibitors, biguanides, D-phenylalanine derivatives, dipeptidyl peptidase-4 (DPP-4) inhibitor, Januvia (sitagliptin phosphate), Meglitinide, Sulfonylureas, Diabinese (chlorpropamide), Glucotrol (glipizide), Glucotrol XL (glipizide long-acting), glynase (glyburide), Micronase (glyburide), Thiazolidinediones Actos (pioglitazone) and Avandia (rosiglitazone), combo pills such as Actoplus Met, Avandamet, Avandaryl, Duetact, Glucovance, Janumet, and Metaglip, SGLT-2 inhibitors, Amylin mimetic Symlin (pramlintide acetate), incretin Byetta (exenatide), GLP-1 receptor agonist, Glyxambi, Segluromet, Steglatro, Steglujan, Synjardy, Xigduo XR, Adlyxin, Basaglar, Ozempic, Ryzodeg, Soliqua, Toujeo, Tresiba, and Xultophy or combinations thereof.

The therapeutic agent can be an antidiarrheal agent, such as attapulgite, bismuth subgallate, bismuth subsalicylate, loperamide, and diphenoxylate; antidotes; antiemetics, such as hyoscyamine, methscopolamine, scopolamine, cyclizine, dimenhydrinate, hydroxyzine, meclizine, promethazine, dronabinol, nabilone, tetrahydrocannabinol, chlorpromazine, prochlorperazine, alosetron, dolasetron, granisetron, ondansetron, palonosetron, aprepitant, fosaprepitant, rolapitant, dexamethasone, metoclopramide, and trimethobenzamide; antigout agents, such as probenecid, sulfinpyrazone, allopurinol, and colchicine or combinations thereof.

Therapeutic agents include antimigraine agents such as dihydroergotamine mesylate, ergotamine tartrate, methysergide maleate, pizotifen maleate, and sumatriptan succinate; antineoplastic agents, including alkylating agents, antimetabolites, mitotic inhibitors, and hormonal agents, or combinations thereof.

Therapeutic agents include antiparkinsonian agents, such as bromocriptine mesylate and lysuride maleate. The therapeutic agent is an antipsychotics, such as chlorpromazine, fluphenazine, perphenazine, prochlorperazine, thioridazine, trifluoperazine, haloperidol, lithium, loxapine, molindone, pimozide, aripiprazole, asenapine, brexpiprazole, cariprazine, clozapine, iloperidone, lurasidone, olanzapine, paliperidone, pimavanserin, quetiapine, risperidone, and ziprasidone. In addition, therapeutic agents include atypical antipsychotics and corticosteroids, such as beclomethasone, betamethasone, budesonide, cortisone acetate, desoxymethasone, dexamethasone, fludrocortisone acetate, flunisolide, flucortolone, fluticasone, propionate, hydrocortisone, methylprednisolone, prednisolone, prednisone, and triamcinolone or combinations thereof.

The therapeutic agent is an anxiolytic agent such as sedatives and/or hypnotics, such as alprazolam, amyiobarbitone, barbitone, bentazeparn, bromazepam, bromperidol, brotizoiam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenuiixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, suipiride, temazepam, thioridazine, triazolam, and zopiclone or combination thereof.

Therapeutic agents also include hypotensive agents, such as amlodipine, carvedilol, benidipine, darodipine, diltiazem, diazoxide, felodipine, guanabenz acetate, indoramin, isradipine, minoxidil, nicardipine, nifedipine, nimodipine, phenoxybenzamine, prazosin, reserpine, and terazosin or combinations thereof.

Therapeutic agents include immunosuppressive agents, miscellaneous therapeutic agents, monoamine oxidase inhibitors, NSAIDs, opiate agonists or opiate partial agonists, such as codeine, dextropropyoxyphene, diamorphine, dihydrocodeine, meptazinol, methadone, morphine, nalbuphine, and pentazocine or combinations thereof.

Therapeutic agents also include respiratory tract agents, skeletal muscle relaxants, thyroid and anti-thyroid agents, such as carbimazole and propylthiouracil; tricyclics and other norepinephrine-reuptake inhibitors, vitamins, wakefulness-promoting agents, sildenafil, tadalafil or combinations thereof.

In some cases, the one or more additional therapeutic agent is selected from an antiviral agent, anti-inflammatory agent, pain reducing agent, and combinations thereof. In some cases, the anti-inflammatory agent is selected from ibuprofen, naproxen, ketoprofen, tometin, etodolac, flurbiprofen, diclofenac, misoprostol, piroxicam, fenoprofen, indomethacin, sulndac, nabumetone, ketorolac, famotidine, mefenamic acid, diflunisal, meloxicam, and combinations thereof. In some cases, the pain reducing agent is selected from ibuprofen, diphenhydramine, acetaminophen, magnesium sailicylate, aspirin, meprobamate, ziconotide, butalbitalor, codeine, hydrocodone, dihydrocodone, oxycodone, naloxone, pentazocine, fentanyl, morphine, hydromorphone, buprenorphine, methadone, meperidine, buprenorphine, oxymorphone, tramadol, nalbuphine, propoxyphene, tapentadol, alfentanil, sufentanil, remifentanil, and combinations thereof.

In some cases, the one or more additional therapeutic agent is trifluridine (TFT), tunicamycin, thapsigargin, Brefeldin-A, or salubrinal. In some cases, the one or more additional therapeutic agent is trifluridine (TFT).

Antiviral Agents

In particular, the disclosure provides for therapeutic agents which are antiviral agents. The disclosure provides for methods of treating or alleviating a viral infection. The term “antiviral agent” refers to an agent that used to inhibit production or replication of viruses that cause disease. Antiviral agents include agents that inhibit transcription of the viral genome such as DNA polymerase inhibitors and reverse transcriptase inhibitors, protease inhibitors which inhibit post-translational events, agents that inhibit the virus from attaching to or penetrating the host cell. Antiviral agents include immunomodulators that induce production of host cell enzymes, which stop viral reproduction, integrase strand transfer inhibitors that prevent integration of the viral DNA into the host DNA by inhibiting the viral enzyme integrase, and neuraminidase inhibitors that block viral enzymes and inhibit reproduction of the viruses.

Exemplary antiviral agents include adamantane antivirals, antiviral boosters, antiviral combinations, antiviral interferons, chemokine receptor antagonist such as CCR5-antagonists, integrase strand transfer inhibitor, miscellaneous antivirals, neuraminidase inhibitors, NNRTIs, NS5A inhibitors, fusion inhibitors, nucleoside reverse transcriptase inhibitors (NRTIs), protease inhibitors, guanosine analog, DNA polymerase inhibitors, guanine nucleotide synthesis inhibitors, and purine nucleosides. In some cases, the antiviral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine, or sofobuvir.

In some cases, the antiviral agent has a concentration ranging from about 10 mg/kg to about 600 mg/kg. In some cases, the concentration of the antiviral agent ranges from about 10 mg/kg to about 100 mg/kg, from about 10 mg/kg to about 200 mg/kg, about 10 mg/kg to about 300 mg/kg, about 10 mg/kg to about 400 mg/kg, about 10 mg/kg to about 500 mg/kg, or about 10 mg/kg to about 600 mg/kg. In some cases, the concentration of the antiviral agent ranges from about 100 mg/kg to about 600 mg/kg, about 200 mg/kg to about 600 mg/kg, about 300 mg/kg to about 600 mg/kg, about 400 mg/kg to about 600 mg/kg, or about 500 mg/kg to about 600 mg/kg. In some cases, the concentration of the antiviral agent is about 100 mg/kg, about 200 mg/kg, about 300 mg/kg, about 400 mg/kg, about 500 mg/kg, or about 600 mg/kg. In some cases, the concentration of the antiviral agent is about 100 mg/kg. In some cases, the concentration of the antiviral agent is about 10 mg/kg.

Methods of Treatment

Provided herein are methods of treating a disease or disorder in a subject, comprising administering to the subject a therapeutically effective amount of 4-phenylbutyrate (PBA) or a pharmaceutically acceptable salt thereof. In some cases, the disease or disorder is a viral infection. In some cases, the viral infection is caused by Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, humanherpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE

In some cases, the disease or disorder is associated with a herpes simplex virus (HSV). In some cases, the disease or disorder is associated with herpes simplex virus 1 (HSV-1) or herpes simplex virus 2 (HSV-2). In some cases, the disease or disorder is associated with herpes simplex virus 1 (HSV-1). In some cases, the disease or disorder is associated with herpes simplex virus 2 (HSV-2). In some cases, the disease or disorder is selected from corneal inflammation, corneal hypertension, corneal sensitivity, corneal dryness, keratitis, ocular herpes, herpes gingivostomatitis, herpes labialis, herpes genitalis, herpetic whitlow, herpes giadiatorurn, herpesviral encephalitis, herpesviral meningitis, herpes esophagitis, neonatal herpes simplex, herpetic sycosis, eczema herpeticum, herpetic keratoconjunctivitis, herpetic uveitis, Sjögren's syndrome, dry eye disease, and combinations thereof.

in some cases, the method comprises administering to a patient a therapeutically effective amount of a composition comprising PBA or a pharmaceutically acceptable salt thereof, and one or more additional therapeutic agents. In some cases, the one or more additional therapeutic agent is selected from an antiviral agent, anti-inflammatory agent, pain reducing agent, and combinations thereof as described herein. In some cases, the method comprises parenteral, nasal, oral, pulmonary, topical, vaginal, rectal, ophthalmic, or sublingual administration. In some cases, the method comprises ophthalmic administration. In some cases, the method comprises prophylactic or therapeutic administration. In some cases, the method comprises therapeutic administration. In some cases, the method comprises administration via preloaded syringe or preloaded dermal (skin) patch. In some cases, the method comprises administration via preloaded syringe. In some cases, the method comprises administration via preloaded dermal (skin) patch.

Also provided are methods of treating an ocular disease or condition in a subject in need thereof, comprising administering to the subject 4-phenylbutyrate (PBA) or a pharmaceutically acceptable salt thereof. In some cases, the method comprises administering to a patient a therapeutically effective amount of a composition comprising PBA or a pharmaceutically acceptable salt thereof, and one or more additional therapeutic agents as disclosed herein. In some cases, the ocular disease or condition is selected from corneal inflammation, corneal hypertension, corneal sensitivity, corneal dryness, ocular herpes, herpetic keratoconjunctivitis, herpetic uveitis, Sjögren's syndrome, keratitis, and combinations thereof.

The disclosure provides for methods of treating respiratory disorders caused by respiratory viruses include the influenza viruses (A and B), H5N1 and H7N9 avian influenza A viruses, parainfluenza viruses 1 through 4, adenoviruses, respiratory syncytial virus A and B and human metapneumovirus, and rhinoviruses (see Table: Some Respiratory Viruses), coronavirus such as Middle East respiratory syndrome coronavirus (MERS-CoV), severe acute respiratory syndrome coronavirus SARS-CoV, and severe acute respiratory syndrome coronavirus 2 (SARS-CoV2). The respiratory disorders include influenza, common cold, MERS, SARS and COVID-19, Influenza, AFRD, acute bronchitis and pneumonia, and croup, to name a few.

The disclosure provides for methods of treating gastroenteritis caused by a virus including Rotavirus, Norovirus, astrovirus, adenovirus 40, adenovirus 41, and coronavirus, to name a few.

The disclosure provides for methods of treating an infection or disorder caused by Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), or coxsackievirus.

Viral infections that can be treated include, at least, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis (encephalitis) (VEE) virus, Chikungunya virus, Western Equine Encephalomyelitis (encephalitis) (WEE) virus, Eastern Equine Encephalomyelitis (encephalitis) (EEE) virus, Tick-borne Encephalitis, Kyasanur Forest Disease, Alkhurma Disease, Omsk Hemorrhagic Fever, Hendra virus, Nipah virus, and species thereof.

In a particular aspect of the disclosure provides for methods of treatment and prophylaxis of herpes infections. e.g. HSV-1 and HSV-2, in particular Herpes simplex infections in patients displaying Herpes labialis, Herpes genitalis, Herpetic gingivostomatitis and Herpes-related keratitis, Alzheimers disease, encephalitis, pneumonia, hepatitis; dermatitis, keratoconjuctivitis, Vulvovaginitis, in patients with a suppressed immune system, such as AIDS patients, cancer patients, patients having a genetic immunodeficiency, transplant patients; in new-born children and infants; in Herpes-positive patients, in particular Herpes-simplex-positive patients, for suppressing recurrence (suppression therapy); patients, in particular in Herpes-positive patients, in particular Herpes-simplex-positive patients.

The disclosure also provides for methods of treating infections caused by human papillomavirus, and the disorders caused by such as warts (verrucae), genital warts, cervical cancer, anogenital cancer and oropharyngeal cancer.

In addition, the methods include treating infections caused by other viruses including Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses.

The disclosure also includes methods of treating an infection caused Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV, Venezuelan equine encephalomyelitis VEE.

The methods of treating a viral infection include methods which reduce of viral load or prevent the viral load from increasing. In particular, the methods reduce viral replication or neutralize the virus. In this way, the disclosed methods prevent the viral load from increasing to a point where it could cause pathogenesis, allowing the body's innate immune mechanisms to neutralize the virus.

Exemplary antiviral agents include maraviroc, enfuvirtide, amantadine, lamivudine, nevirapine, efavirenz, dolutegravir, elvitegravir, raltegravir, acyclovir and any nucleoside analog of aciclovir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine and sofobuvir. Other exemplary antiviral agents include peptide based compounds that can target viral/non-viral targets and DNA based compounds that can target viral/non-viral targets.

### EXAMPLES

Materials and Methods:

4PBA and sodium PBA were purchased and used as-is from BioVisioin (1608-1000). ACV, TFT, gangiclovir, valacyclovir, famcyclovir, and pencyclovir were purchased from Selleck Chemicals.

Cells and Viruses

Human corneal epithelial cells (RCB1834 HCE-T) were obtained from Kozaburo Hayashi (National Eye Institute, Bethesda, MD) and were cultured in minimum essential medium (MEM) (Life Technologies, Carlsbad, CA) with 10% fetal bovine serum (FBS) (Sigma-Aldrich, St. Louis, MO) and 1% penicillin/streptomycin (P/S) (Life Technologies The human vaginal epithelial cell VK2/E6E7 was obtained from the ATCC. VK2/E6E7 cells were passaged in keratinocyte serum-free medium (KSFM) (Gibco/BRL, Carlsbad, CA) supplemented with epidermal growth factor (EGF), bovine pituitary extract (BPE), and 1% penicillin-streptomycin. African green monkey kidney (Vero) cells were provided by P. G. Spear (Northwestern University). Madin-Darby bovine kidney (MDBK) cells were kindly provided by Prof. Richard Longnecker's lab from Northwestern University. Vero and MDBK cells were passaged in Dulbecco's modified Eagle's medium (DMEM) supplemented with 10% FBS and P/S.

HSV-1 (17 GFP), HSV-2 (333 GFP), and β-galactosidase-expressing HSV-1 (gL86), HSV-2 (333) used in this study were kindly provided by Patricia Spear's lab at Northwestern University. Virus stocks were propagated and titered on Vero cells, and stored at −80° C. PRV and BHV were kindly provided by Profs Greg Smith and Richard Longnecker respectively from Northwestern University and propagated, and titered on MDBK cells.

MTT Assay

MTT viability assay using various concentrations of PBA/NaPBA were performed on HCE cells plated at a density of 1×104 per well in a 96 well plate overnight (54). Concentrations starting at 100 mM were two-fold serially diluted in whole media and added to cell monolayers for a period of 24 h. At the end of incubation, MTT (0.5 mg/mL in whole media) was added to cells and incubated for a period of 3 hours to allow crystal formation. Acidified isopropanol (1% glacial acetic acid v/v) was added to cells to dissolve the formazan crystals. Dissolved violet crystals were transferred to a new 96 well plate and analyzed by a micro-pate reader (TECAN GENious Pro) at 492 nm.

Viral Entry Assay

β-Galactosidase expressing viruses HSV-1 gL 86 at MOI 10 was used in this study. HCEs were plated at a density of 1×104 in 96 well plates overnight before use. HSV-1 strain gL86 was mixed with multiple concentrations of PBA/NaPBA in MEM media and overlaid on HCEs to infect the cell monolayers for 6 h after which the cells were washed with PBS twice and 1004 of soluble substrate o-nitrophenyl-β-d-galactopyranoside (ONPG) 3 mg/mL was added to the cells along with 0.5% Nonidet β-40 in PBS. Enzymatic activity was measured by a micro-pate reader (TECAN GENious Pro) at 405/600 nm.

Transfections:

All transfection experiments were performed according to manufacturer's protocols. Typically in a plasmid transfection experiment, all plasmids were procured from Addgene, OptiMEM was used as the transfection medium and Lipofectamine 2000 was used as the reagent. In CREB3 siRNA knockdown experiments, the siRNA was purchased as a pre-validated set of 3 siRNAs from Integrated DNA technologies (IDT), OptiMEM was used as the transfection medium and RNAi-Max Lipofectamine was used as the transfection reagent. All transfections were performed for a period of 48 hours, with media changed at 12 hours post transfection.

Immunoblotting

Cells were scraped and incubated with 1004 of Radioimmunoprecipitation assay (RIPA) buffer with protease phosphatase inhibitor cocktail (Halt™, 78440) for a period of 30 minutes on ice. Whole cell protein extracts (supernatant) were collected by centrifuging the mixture at 13,500 rpm on a bench top refrigerated (4° C.) centrifuge for 15 minutes. Protein samples were then denatured in NuPAGE LDS Sample Buffer (Invitrogen, NP00007) and 6-mercaptoethanol by heating them to 80° C. for 10 min. The denatured protein samples were allowed to cool and equal amounts of protein were added to 4-12% SDS-PAGE loading gels and run at a constant speed of 70V for 3 h. The protein from the gel was then transferred to a nitrocellulose membrane using an iBlot 2 dry transfer machine (Thermofisher Scientific, USA). Nitrocellulose membrane was blocked in 5% nonfat milk/BSA in tris buffer saline (TBS) and 0.1% tween 20 (TBST) for 1 h at room temperature. After the blocking step, membranes were incubated with primary antibody at dilutions of 1:1000 overnight at 4° C. The following day the blots were washed multiple times with TBST before the addition of horse radish peroxidase conjugated secondary IgG antibody at dilutions of 1:10,000 at room temperature. Anti-HSV-1 gB mouse monoclonal antibody (abcam, 6506) and anti-GAPDH (Proteintech, 10494-1-AP) was used to evaluate extent of viral infection. All ER Stress related proteins referred to in this manuscript were purchased from Cell Signaling as an ER Stress Antibody Sampler Kit (CST #9956). Luman/CREB3 was purchased from Proteintech (11275-1-AP). Primary antibodies from Cell Signaling and Proteintech were used at 1:1000 dilution and secondary antibodies at 1:2000 dilution. Protein bands were visualized on an ImageQuant LAS 4000 imager (GE Healthcare Life Sciences) by the addition of SuperSignal West Pico maximum sensitivity substrate (Pierce, 34080). The density of the bands was quantified using ImageQuant TL image analysis software (version: 7). GAPDH was measured as a loading control.

Cytoplasmic and Nuclear Extractions

The cytoplasmic and nuclear extractions were performed using a protocol previously described (Hadigal et al 2015, Nat Comm). Aliquots of the fractions were made used for immunoblotting as described above.

Plaque Assay

Infected cells suspended in 5004 of Opti-MEM prior to sonication using a probe sonication system at 70% amplitude for 30 seconds. Sonicated cell lysates or ocular/vaginal swabs were used as inoculants for a plaque assay to determine the total amount of infectious virus. In a typical experiment, Vero cells were plated at a seating density of 5×104 per well in a 24 well plate overnight. Upon confluency, the cell monolayers were washed with PBS and infected samples were overlaid for a period of 2 h at multiple dilutions. The cells were then washed twice with PBS prior to the addition of DMEM mixed with 0.5% methylcellulose. The plates were incubated for 72 h at 37° C. and 5% CO2 before they were fixed with methanol and stained with crystal violet to determine the extent of plaque formation.

Quantitative PCR Assay

RNA from cells was extracted using Trizol (Life Technologies) according to manufacturer's protocol. Extracted RNA was quantified using Nanodrop (Thermofisher Scientific, USA) and equilibrated for all samples with Molecular Biology Grade water (Corning, USA) before they were reverse transcribed into cDNA using High Capacity cDNA Reverse Transcription Kit (Applied Biosystems, Foster City, CA). Equal amounts of cDNA were analyzed via real-time quantitative PCR using Fast SYBR Green Master Mix on QuantStudio 7 Flex system (Applied Biosystems). The primers used in this study are as follows: ICP-0 F—GTG CTG CGC CAA GAA AAT (SEQ ID NO: 1), ICP-0 R—TCA ACT CGC AGA CAC GAC TC (SEQ ID NO: 2), gD F—TAC AAC CTG ACC ATC GCT TC (SEQ ID NO: 3), gD R—GCC CCC AGA GAC TTG TTG TA (SEQ ID NO: 4), ATF-4 F—CCC TCC AAC AAC AGC AAG GA (SEQ ID NO: 5), ATF-4 R—ACC CAA CAG GGC ATC CAA G (SEQ ID NO: 6), CHOP F—CAT CAC CAC ACC TGA AAG CAG A (SEQ ID NO: 7), CHOP R—TGG ACA GTG TCC CGA AGG AG (SEQ ID NO: 8), GRP78 F—CAA CCA AAG ACG CTG GAA CTA TT (SEQ ID NO: 9), GRP78 R—CCC TCT TAT CCA GGC CAT AAG C (SEQ ID NO: 10), GRP94 F—TCA GAG ACA TGC TTC GAC GAA (SEQ ID NO: 11), GRP94 R—CTG ACC GAA GCG TTG CTG T (SEQ ID NO: 12), GAPDH F—TCC ACT GGC GTC TTC ACC (SEQ ID NO: 13), GAPDH R—GGC AGA GAT GAT GAC CCT TTT (SEQ ID NO: 14).

Confocal Microscopy Imaging

Fluorescent confocal imaging was performed on a LSM 710 confocal microscope (Carl Zeiss) under 63× objectives. Briefly, cells that were cultured and experimented in glass bottom dishes (MatTek Corporation) were washed in PBS, fixed with 4% paraformaldehyde (PFA, Electron Microscopy Sciences, Hatfield, PA) for 10 mins then washed with PBS again. A permeabilization step was performed using 0.01% Triton-X (Fisher Scientific) for 10 mins after fixing the cells followed by a blocking step for one hour in 1% Bovine Serum Albumin (BSA, Sigma-Aldrich). Cells were then incubated with the primary antibody in 1% BSA for 1 hr. Following washes, the cells were incubated with a secondary-conjugated Alexa-fluor-647 in 1% BSA for 1 hr.

Flow Cytometry

Cells infected with GFP virus were collected 24 hpi using Hank's based, enzyme-free cell dissociation buffer (Gibco-13150) (58, 59). Cells were washed once with PBS before they were fixed using 4% parafolmaldehyde (Electron Microscopy Sciences, USA) and suspended in fluorescence-activated cell sorting (FACS) buffer (PBS, 5% FBS). Cell suspensions were filtered through a 70-μm mesh, resuspended in FACS buffer before analyzing them using an in-house flow cytometer (BD Accuri C6 Plus). A total of 2×104 cells were collected for each sample and the analysis was performed using FlowJo (version 10).

Isolation of Primary Human Corneal Epithelial Cells

Donated human corneas were gently washed and the iris from the sclera was removed prior to placing them epithelium down into 1% dispase solution overnight at 4° C. Next day, using a blunt spatula, cells were dislodged from each of the corneas in PBS. Collected cells were centrifuged and resuspended in 1 mL of 0.5% Trypsin for 30 seconds. Trypsin was neutralized with help of 10% FBS solution in PBS and the cells were centrifuged at 800 g for 5 minutes. Collected cells were resuspended in keratinocyte media with 1% penicillin/streptomycin and 10% FBS. The cells were then allowed to grown in a pre-coated flask (Gibco Coating matrix) for 2 weeks. The first 2 passages of cells were discarded and only the cells from the 3nd passage were used for the experiments.

### Example 7

### CREB3 is Upregulated Upon HSV-1 Infection

Human corneal epithelial cells were either mock infected of infected with 0.1 MOI HSV-1 17 GFP. At 24 hpi. cells were collected and immunoblotted for shown proteins (FIGS. 1A and 1C). In a similar experiment, cells plated in a glass bottom dish were fixed with 4% PFA and stained for CREB3 (Red), HSV-1 (Green) and nucleus (blue) (FIG. 1B). In a time course experiment, HCEs were infected with 0.1 MOI HSV-1 17 GFP for 0, 3, 6, 12, 24 or 48 hours and collected for immunoblotting analysis or quantitative reverse transcription PCR (FIGS. 1D and 1E). HSV-1 infected HCEs were collected 24 hpi and the downstream effectors of CREB3 were analyzed through qRT-PCR and fluorescent imaging (FIGS. 1F and 1G). Primary human corneal epithelial cells were isolated from donated human corneas and infected with HSV-1 17 GFP at 0.1 MOI for 24 hours prior to immunoblotting and imaging them for the presence of CREB3 (FIGS. 1H and 1I). A gene ontology diagram of CREB3 showing its role in unfolded protein response and negative regulation of ER-stress induced apoptosis (FIG. 1J). HCEs were transfected with GFP or HPSE-GFP overexpression plasmid for 24 hours, prior to the addition of mock or 0.1 MOI HSV-1 Kos (FIG. 1K). The cells were collected 24 hpi and immunoblotted for shown proteins. Transfection efficiency and expression of HPSE-GFP can be seen via fluorescent images (FIG. 1L).

### Example 8

### Modulation of CREB3 Expression Effects Viral Life Cycle

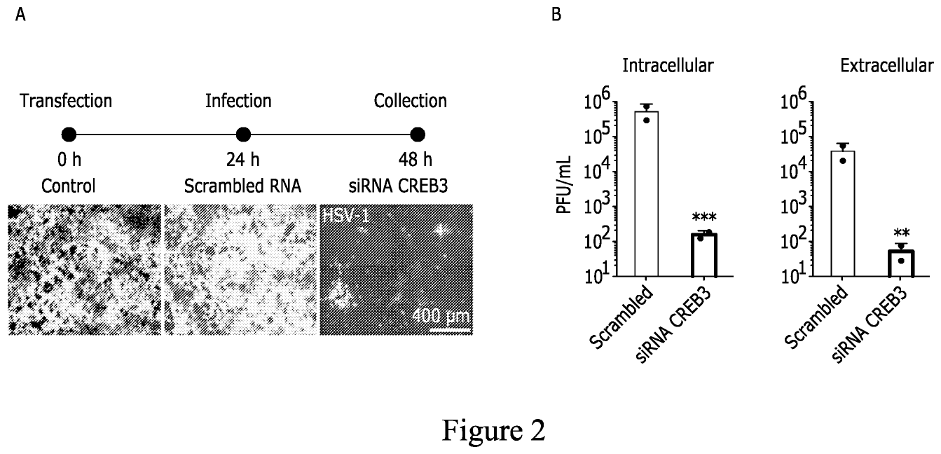

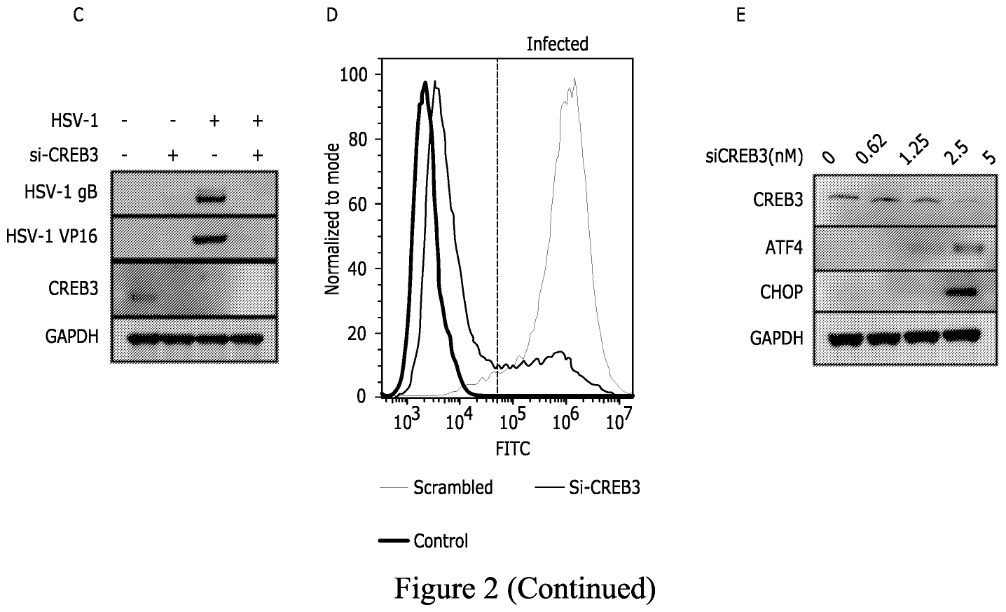

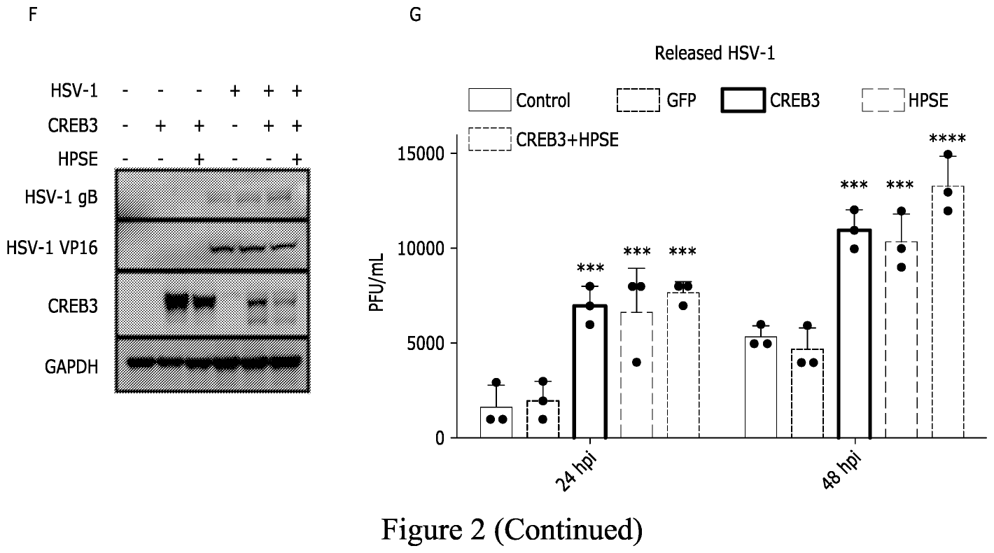

HCEs were transfected with CREB3 siRNA for a period of 24 hours prior to the addition of 0.1 MOI HSV-1 17 GFP. At 24 hpi, cells were imaged using a fluorescent microscope, cell lysates and supernatant from the cell culture were used as inoculum for a plaque assay on Vero cells, cell lysates were used for immunoblotting analysis, and flow cytometry analysis to quantify extent of viral infection (more green fluorescence=more infection) (FIGS. 2A-2D). HCEs were transfected with increasing concentrations of CREB3 siRNA and immunoblotted to evaluated changes in ATF4 and CHOP along with GAPDH as the loading control (FIG. 2E). HCEs were transfected with CREB3 overexpression plasmid for a period of 24 hours prior to infecting them with mock or 0.1 MOI HSV-1 17 GFP (FIG. 2F). At 24 hpi. cell lysates were immunoblotted for requisite proteins, cell culture supernatant was collected and used as inoculum to assess released virus via plaque assay on Vero cells (FIG. 2G).

### Example 9

### ER Stress Alleviation Through PBA Reduces Viral Infection and Induces CHOP and ATF Expression

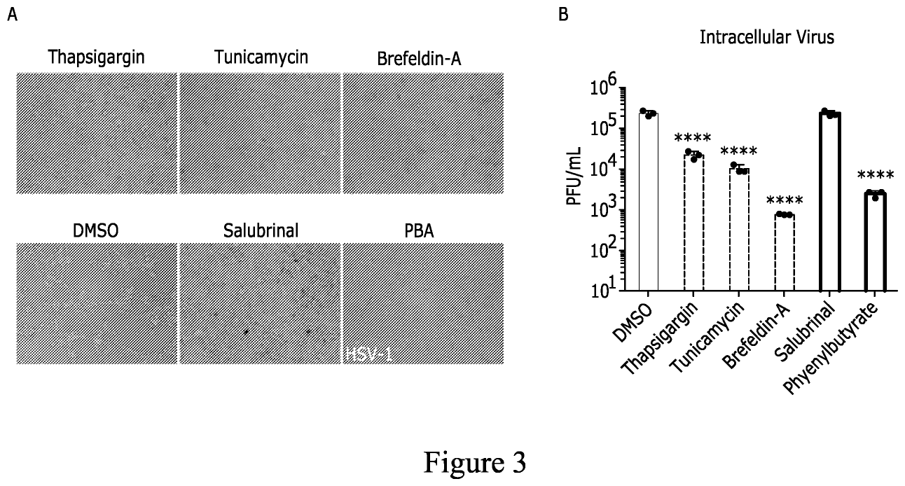

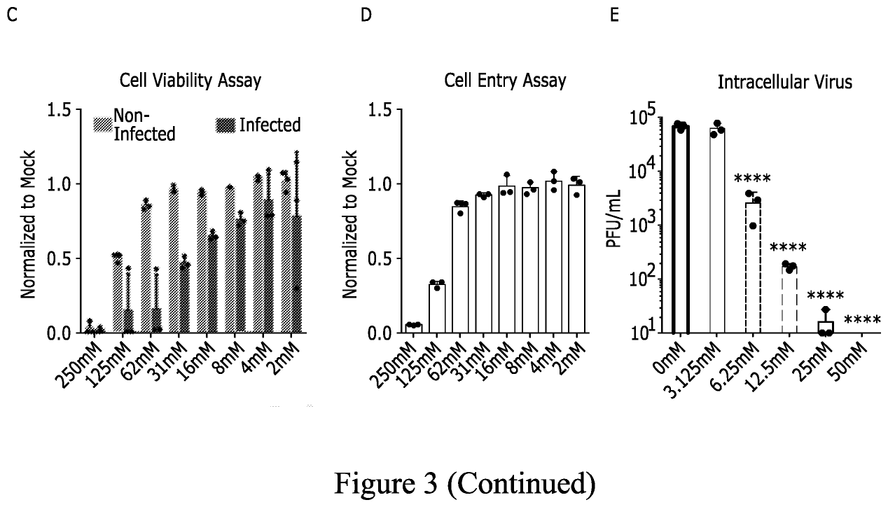

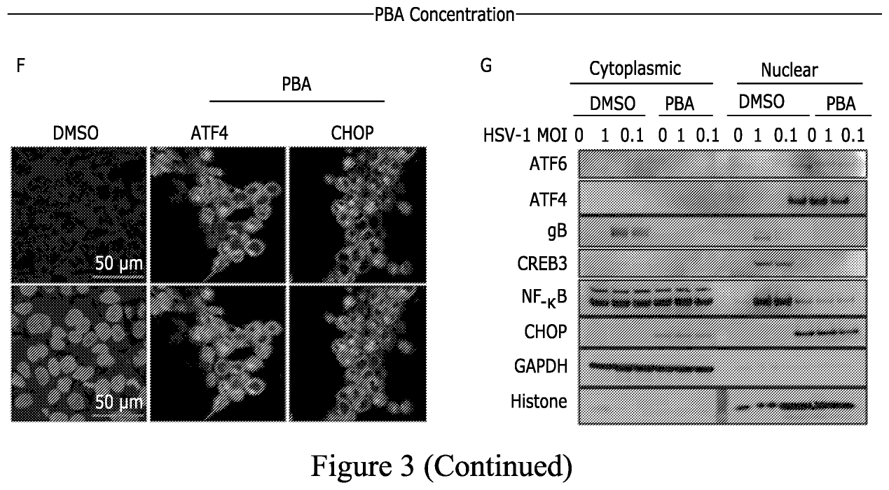

HCEs were infected with 0.1 MOI HSV-1 K26RFP and treated with tunicamycin (1 μM), thapsigargin (1 μM), Brefeldin-A (1 μM), salubrinal (75 μM) or PBA (10 mM). At 24 hpi, cells were imaged for the presence of fluorescent HSV-1 and cells were lysed and used as inoculum on Vero cells to assess viral titer via plaque assay (FIGS. 3A and 3B). HCE viability in the presence of various concentration of PBA under non-infectious and infectious conditions was determined via MTT assay (FIG. 3C). The effect of PBA on viral entry was assessed through a 6-galactosidase producing reporter HSV-1 gL86 using ONPG as the substrate (FIG. 3D). Phenylbutyrate treatment is well tolerated and does not inhibit viral entry. HCEs infected and treated with PBA with various concentrations were collected 24 hpi and the lysates from these cells were used as inoculum on Vero cells to determine a concentration dependent viral inhibition via a plaque assay (FIG. 3E). HCEs plated in glass bottom dishes were treated with Mock DMSO or PBA (10 mM) for 24 hours prior to fixing them with 4% PFA and staining them with fluorescently labelled antibodies (FIG. 3F). These dishes were imaged at 63× on a Zeiss Confocal microscope (FIG. 3G). HCEs were infected at MOI of 0, 1 or 0.1 for 24 hours in the presence or absence of PBA. Cells were lysed in a hypotonic buffer to remove the cytoplasmic protein portion followed by RIPA buffer to extract nuclear proteins. The samples were separated and immunoblotted for shown proteins.

### Example 10

### Therapeutic Efficacy of PBA in Ex Vivo Human and In Vivo Murine Tissues Infection of Human Corneas

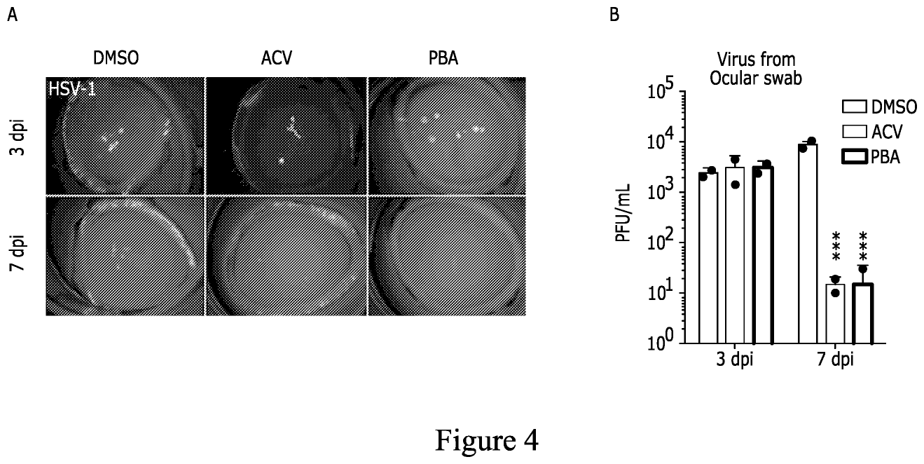

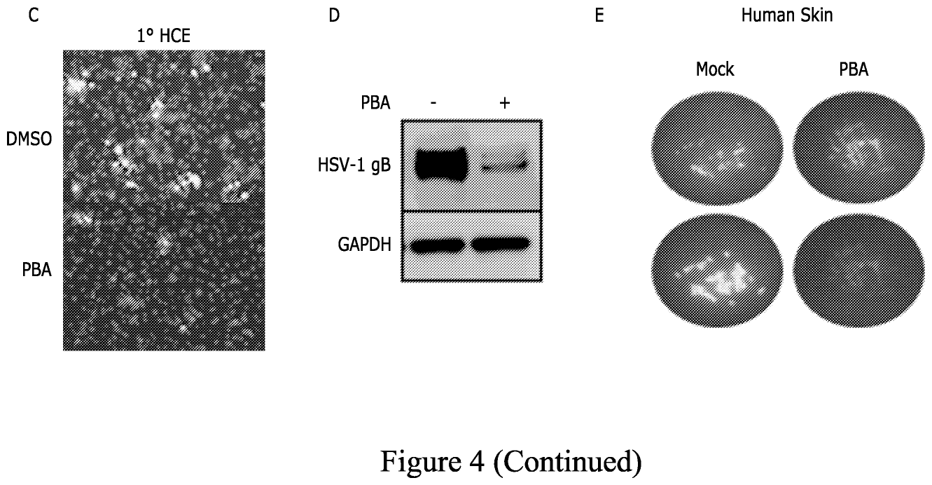

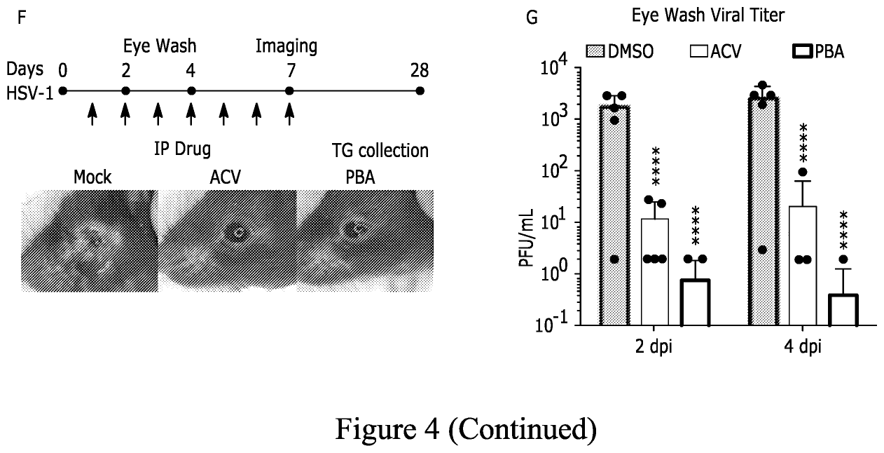

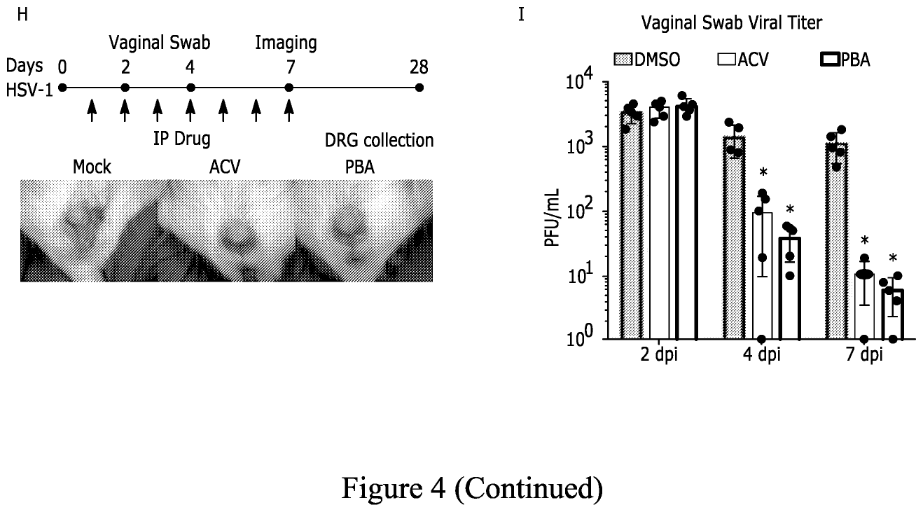

Human corneas (5 per group; 3 groups) were washed gently in PBS and cultured in a 12 well-plate. Epithelial debridement was performed on the center portion of the cornea using a 30G needle followed by infection with HSV-1 17 GFP. It was previously found that depending on length of duration between cornea isolation from the human and final use, all human corneas get differentially infected. In order to avoid bias, only similarly infected corneas (2 per group) were chosen to conduct this experiment. The virus was allowed to propagate for 3 days before adding ACV (50 μM), PBA (10 mM) or DMSO control to the cornea culture media. At 3 dpi, GFP fluorescence from infected corneas was visualized using a Zeiss Stereoscope. Starting 3 dpi, treatments including ACV (50 μM), PBA (10 mM) or DMSO control in cornea media (Gibco MEM with 1% Antibacterial: Antifungal-Gibco and 10% FBS-Sigma US origin). The media was changed every day until 7 dpi when the extent of infection was visualized again (FIG. 4A). Ocular swabs taken on 3 dpi and 7 dpi were quantitated for the presence of virus using a plaque assay (FIG. 4B).

Primary human corneal epithelial cells were isolated from donated human corneas and cultured in a tissue culture flask (FIG. 4C). On the third passage, cells were plated and infected with HSV-1 17 GFP followed by treatment with PBA (10 mM) or DMSO control. At 24 hpi, images were taken at 10× using a fluorescent microscope. Cell lysates were immunoblotted for the presence of viral proteins (FIG. 4D).

Human Skin Graft Infection

Human skin grafts were purchased from Genoskin. The HypoSkinR model consists of adipose tissue for subcutaneous injection, topical and systemic administration in a ready-to-use format. The epithelial layer of the skin was abraded using a sterilized blade and HSV-1 17 GFP was added topically. The skin grafts were checked every day for progression of viral replication (increase in GFP fluorescence). Starting 3 dpi, either PBA (20 mM) or DMSO control were dissolved in the skin media (provided by the supplier) and added to the bottom of the Genoskin plate. This represents a systemic model of drug administration. The medicated media was changed every day and images were procured 7 dpi. The fluorescence was quantified over the treatment period.

Human skin samples procured from GenoSkin were cultured according to manufacturer's protocol (FIG. 4E). Skin epithelium was abraded using a sharp blade and infected with HSV-1 17 GFP. At 3 dpi, fluorescent image of the skin was acquired using a Zeiss stereoscope. PBA was administered to the skin systemically by adding PBA (10 mM) to the growth media provided by the manufacturer. At 7 dpi, fluorescent images were taken again to assess extent of viral spread in the skin samples.

Ocular Toxicity Studies

All the ocular toxicity studies including surface dryness, optical coherence tomography, corneal sensitivity and ocular hypertension were performed similar to methods mentioned previously by our lab (Yadavalli et al 2019, The Ocular Surface). Drugs were administered 3 times every day for 4 weeks and the readings were taken every 2 weeks.

Eight week old male C57BL6 mice (n=5 per group) were infected with 5×10⁵ PFU HSV-1 McKrae after corneal epithelial debridement with a 30G needle (FIG. 4F). At 1 dpi, ACV (5 mg/kg), PBA (50 mg/kg) or PBS mock were administered to mice twice a day intraperitoneally with volume not exceeding 300 μL per dosing. Murine eyes were imaged using a Zeiss stereoscope to assess the extent of disease progression on 7 dpi. Ocular swabs were collected on days 2 and 4 dpi and used as inoculum on Vero cells to determine viral titers using a plaque assay (FIG. 4G).

In Vivo Vaginal Infection and Treatment

Naïve 4-week-old female BALB/c mice were purchased from Jackson Laboratory (Bar Harbor, USA) and housed in the university BRL for a period of one week for acclimatization before they were subcutaneously injected with 2 mg of medroxyprogesterone (Depo-Provera) per mice (˜20 g). 5 days later, mice were intravaginally infected with 5×105 PFU HSV-2 (333 strain). Similar to the ocular model of infection drugs were administered intraperitoneally starting 1-day post infection (60). Vaginal swabs were collected using a Calgiswab (Calcium Alginate Mini-tip Urethro-Genital Swab, Puritan) dipped in OptiMEM (Gibco, USA). Images of the ano-genital region were taken on day 7 post infection using a Carl Zeiss stereoscope at 7.5× magnification.

The menstrual cycle of eight week old female BALB/C mice were synchronized using medroxy-progesterone 5 days prior to infecting them with 5×10⁵ PFU HSV-2 333 (FIG. 4H). Animals were treated with ACV (5 mg/kg), PBA (50 mg/kg) or PBS mock were administered to mice twice a day intraperitoneally with volume not exceeding 3004 per dosing. Infected genitalia were imaged using a Zeiss Stereoscope on 7 dpi. Vaginal swabs collected on 2, 4 and 7 dpi were used as inoculum on Vero cells to assess the viral titer using plaque assay (FIG. 4I).

Experiments involving animals were performed under a UIC approved protocol ACC 14-091. The mice were monitored for weight loss and disease scores were recorded in a blinded fashion for 14 days. Sick mice were euthanized according to the IACUC protocol followed by the collection of ocular/vaginal tissue and lymph nodes. Ocular wash and vaginal swabs were used to assess viral titers using a plaque assay.

### Example 11

### PBA Synergizes with TFT to Reduce Effective Antiviral Concentration and Ocular Toxicity

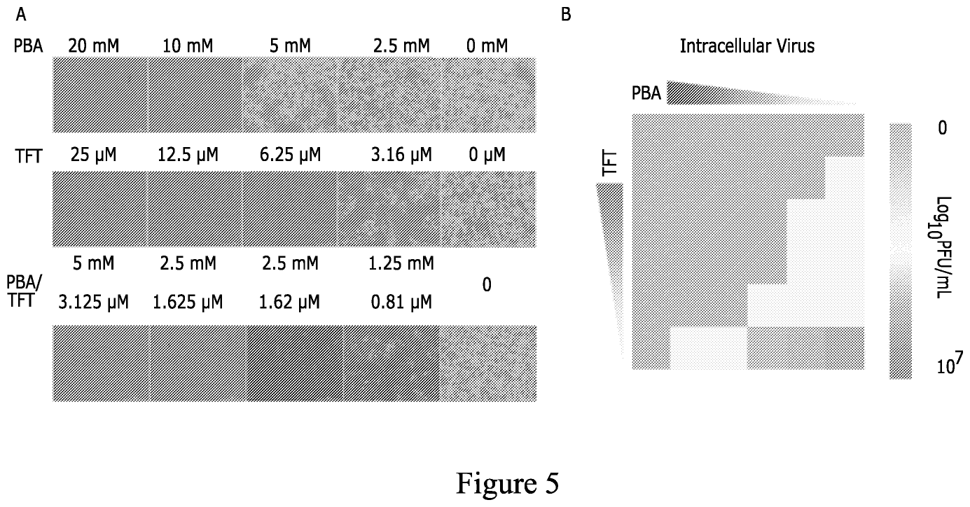

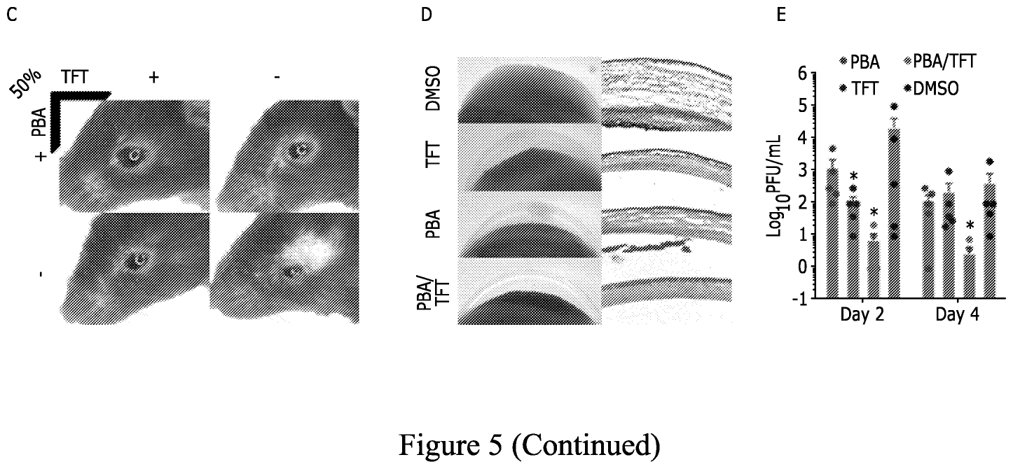

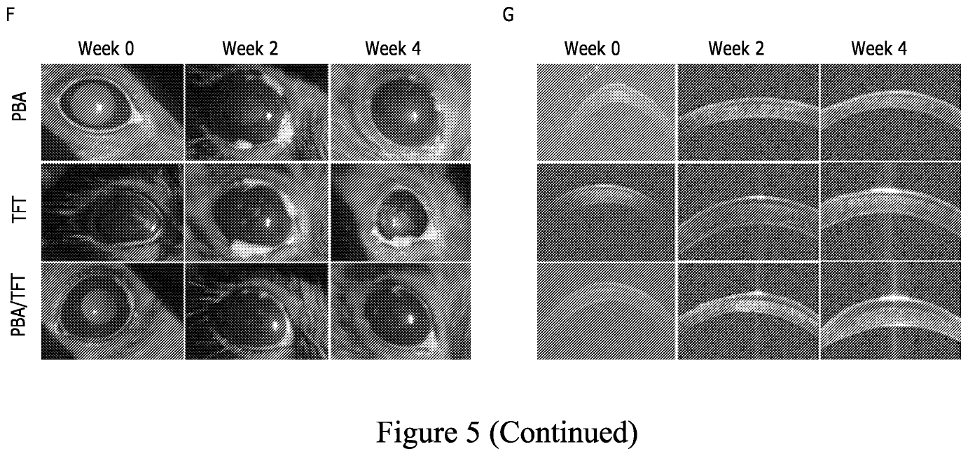

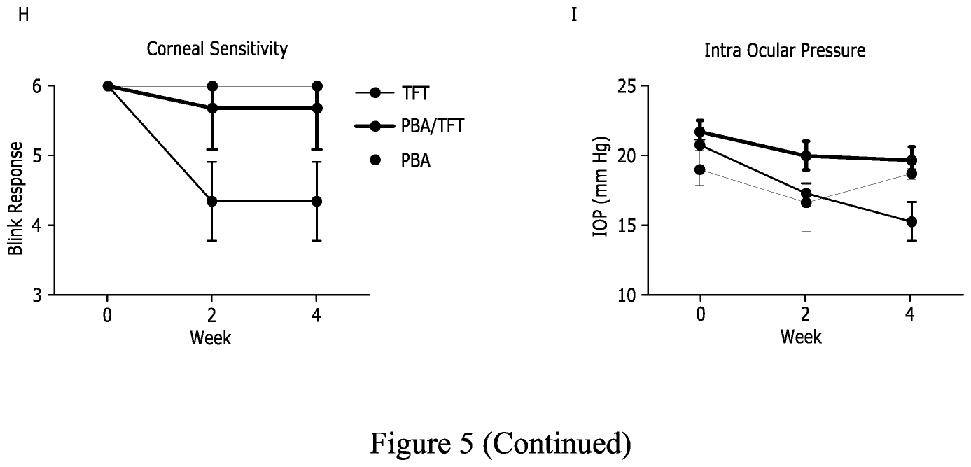

HCEs infected with K26 RFP were treated with PBA alone, TFT alone or PBA+TFT cocktail at shown concentrations. The cells were imaged using a fluorescent microscope at to assess the extent of viral spread (FIG. 5A). Cell lysates were used as inoculum on Vero cells to assess the viral titer through plaque assay (FIG. 5B). PBA concentration was varied from 20 mM to 1.25 mM with 2 fold dilution per step while TFT was used from 6.25 to with 2 fold dilution per step.

In Vivo Ocular HSV-1 Infection and Treatment

C57BL/6 mice (n=5) bred and housed at the university biological resource laboratory (BRL) were used for ocular model of murine HSV-1 infection. Standard feed and water were provided to the mice with a 12 h light and dark cycle with no more than 5 mice per cage. On day 0, 6 to 8-week-old mice were anesthetized, as described previously, prior to the application of a topical anesthetic (proparicaine hydrochloride, 0.5%). Corneal epithelial debridement was performed using a 30 G needle followed by the application of PFU HSV-1 (McKrae) to the eye. Intraperitoneal injections of ACV (5 mg/kg), NaPBA (50 mg/kg) or vehicle alone dispersed in 2% DMSO, 30% PEG-200 and 2% Tween 80 dissolved in PBS were administered starting 1-day post infection. Ocular swabs, disease scores and stereoscopic images (Carl Zeiss Stereoscope) were collected on days 2, 4, 7 and 10 post infection. 28 days post infection, the mice were euthanized, followed by cervical dissociation and their trigeminal ganglia were incubated in DMEM whole media. 4 days post incubation, the supernatants from the trigeminal ganglia suspension was titered for viral reactivation from using a plaque assay on Vero cells.

Eight week old male C57BL6 mice were infected with 5×10⁵ PFU HSV-1 McKrae after corneal epithelial debridement (FIG. 5C). Starting 1 dpi, murine eyes were dosed with TFT (25 μM), PBA (5 mM), PBA (5 mM)+TFT (25 μM) cocktail or DMSO control, 3 times a day for 7 days. Murine eyes were imaged using a Zeiss stereoscope to assess the extent of disease progression on 7 dpi. Mice euthanized on 7 dpi and they were imaged directly (left) or cryo-sectioned and stained with H&E to visualize the extent of ocular tissue damage (FIG. 5D). Ocular swabs taken 2 and 4 dpi were used as inoculum on Vero cells to assess the viral titer using plaque assay (FIG. 5E). In a separate experiment, 8-week old C57BL6 mice were dosed every day with TFT (25 μM), PBA (5 mM), PBA (5 mM)+TFT (25 μM) cocktail or DMSO control, 3 times a day for 4 weeks (FIG. 5F). Slit lamp fluorescent images were acquired every 2 weeks after instilling fluorescein on the eye to detect surface dryness. Optical coherence tomography was also conducted on anesthetized mice every 2 weeks to assess corneal inflammation (FIG. 5G). Corneal sensitivity through blink response using an esthesiometer was performed every 2 weeks on wake mice (FIG. 5H). Intra ocular pressure was measured using a hand held murine tonometer on anesthetized mice to measure ocular hypertension (FIG. 5I).

### Example 12

### Synergizing Acyclovir with PBA can Reduce the Effective Dose Required to Control HSV-1 Encephalitis

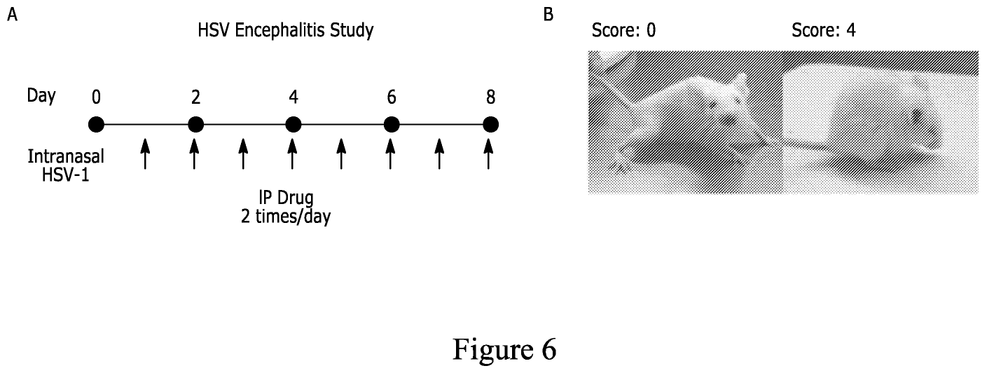

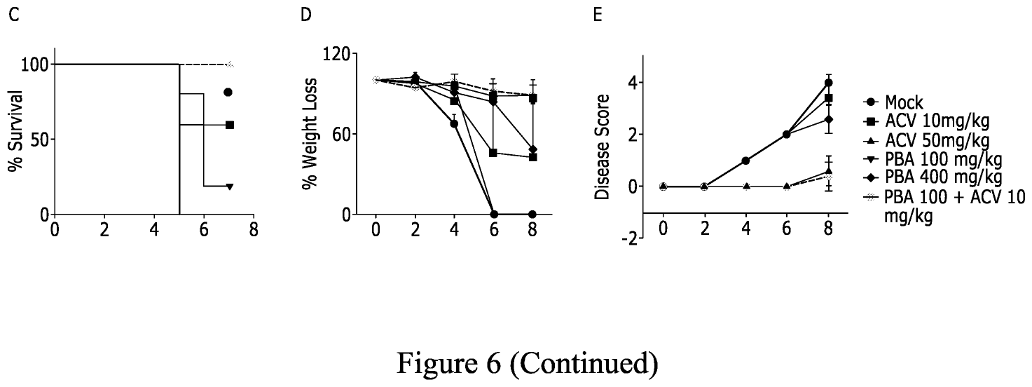

Eight week old female BALB/C mice were infected with 1×105 PFU HSV-1 McKrae through intranasal route to induce encephalitis (FIG. 6A). Infected mice when treated with ACV look healthier with no behavioral changes (left) compared those left untreated (right) (FIG. 6B). Infected mice were administered ACV (50 and 10 mg/kg), PBA (100, 400 mg/kg) or a cocktail of PBA (100 mg/kg)+ACV (10 mg/kg) (FIG. 6C). Percentage survival was calculated 10 dpi for each group based on survival criteria (no more than 20% weight loss). Animal weights were measured daily and animals showing acute weight loss or behavioral changes were euthanized for humane reasons (FIG. 6D). A blinded reviewer was used to assess the disease progression and score the mice on each day (FIG. 6E).

HSV-1 Encephalitis Experiment:

To cause encephalitis in mice, 8 week old Balb/C mice were inoculated intranasally with 5×105 PFU HSV-1 (McKrae). The procedure was performed on anesthetized mice using a sharp ultra-long thin 10 μL micropipette tip in right nostril. All the drugs requisite for this experiment were prepared in advance and stored at 4° C. for later usage. ACV sodium was prepared in 2 different concentrations (50 mg/kg and 10 mg/kg); PBA was prepared in 2 different concentrations (400 mg/kg and 100 mg/kg); ACV+PBA cocktail was prepared in one concentration (ACV-10 mg/kg and PBA 100 mg/kg). All the drugs were formulated in such a way that requisite dosage to each mice could be administered in 25 μL of PBS. Drugs were administered starting 1 dpi, twice a day for 10 days. Weights and disease scores were monitored every day until 10 dpi. Mice with acute weight loss (>20%) and behavioral changes were euthanized for humane reasons. Results are shown in FIG. 6.

### Example 13

### HSV-1 Encephalitis Keratitis Resolution with PBA

Previous studies have shown that infection with HSV-1 induces cell mediated, humoral and intrinsic immune response which when not controlled efficiently can lead to severe tissue damage and in rare circumstances death. While nucleoside analogs such as acyclovir are very effective at controlling viral replication, they do not contribute directly to the amelioration of inflammatory response. Previous studies on PBA have hinted towards its anti-inflammatory potential by alleviating LPS induced inflammatory death in lung epithelia and presenting therapeutic effect on osteoarthritis models. There are no literature reports the effectiveness of PBA on HSV-1 induced corneal inflammation or keratitis.

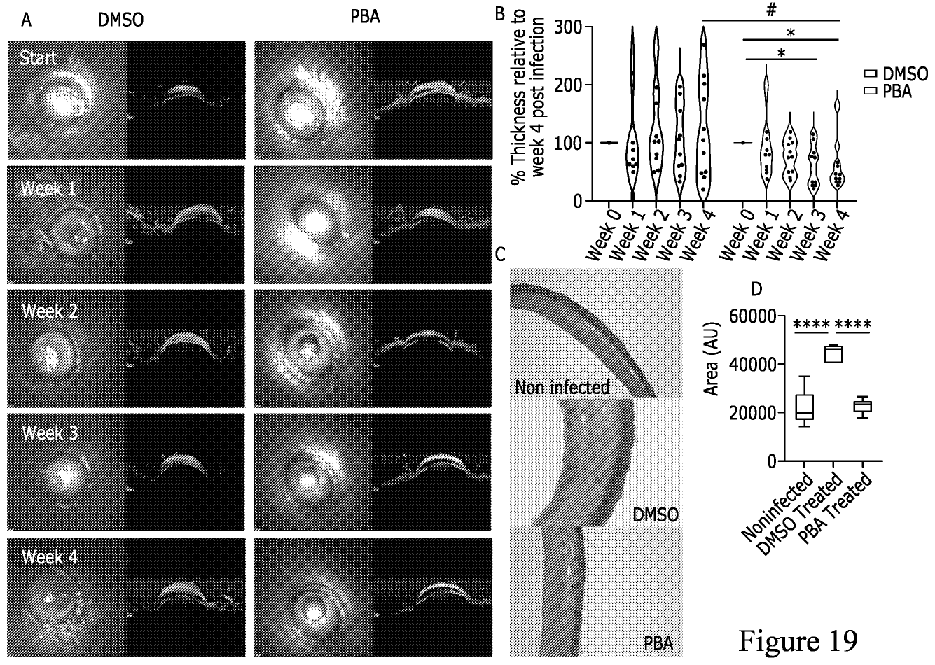

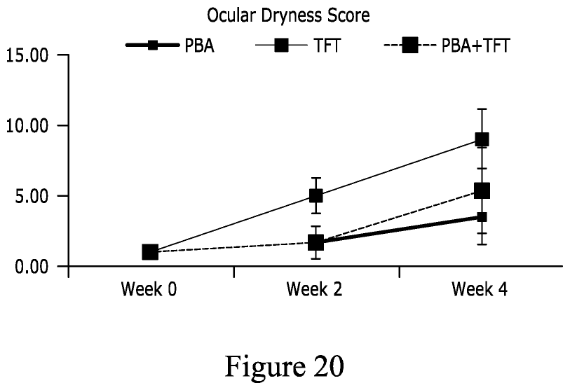

An experiment was performed to ascertain the effectiveness of PBA in preventing or reducing HSV-1 induced inflammation of the eye. Mice were first infected on the right with a low enough viral titer of HSV-1 (1×10⁴ PFU) which induced viral keratitis without causing any other deleterious effects. At 28 days post infection, mice with positive corneal keratitis were separated into 2 groups and one was treated with PBA (5 mM in PBS), while the other was treated with PBS alone for 4 weeks. Animals were dosed 3 times a day every day on their right eye and progression of inflammation was monitored once weekly via optical coherence tomography. Results indicate a significant decrease in corneal thickness (a known marker for corneal inflammation) in PBA treated mice during the 4 week treatment period (FIG. 19).

In Vivo Herpetic Keratitis Model and Treatment

Procedures to care for the mice and maintenance of murine colonies were performed according to a protocol approved by the Animal Care Committee at the University of Illinois at Chicago. The mice were bred and housed at the biologic resource laboratory at UIC. 6-10 week old C57BL/6 mice were used for animal experiments. Mice were given food and water ad libitum, and cage sizes were limited to 5 mice maximum. On day 0, n=50 mice were anesthetized with an intraperitoneal injection of ketamine (100 mg/kg) and xylazine (5 mg/kg). Afterwards, they were given 0.5% proparacaine hydrochloride topically. Corneal epithelial debridement in a 3×3 grid pattern was performed using a 30-guage needle. 1×105 PFU/mL HSV-1 McKrae was applied immediately afterwards to the ocular surface. Mice were untreated for 4 weeks following infection to develop herpetic keratitis. A subset of the mice (n=22) developed keratitis, while the other mice (n=28) either did not develop keratitis or had severe ulceration of the cornea. The mice with mild keratitis were divided into two groups (n=11 each) and treated with 5 mM PBA or DMSO topically three times per day. OCT images were taken on a SPECTRALIS OCT module (Heidelberg Engineering) at 4 weeks post infection and weekly for another four weeks. Images were analyzed using ImageJ to assess corneal thickness. At 8 weeks post infection and 4 weeks post treatment, the mice were euthanized, and whole eye tissue was collected and embedded in optimal cutting temperature (OCT) compound for sectioning.

Immunohistochemistry

Embedded whole eye samples were sectioned using an NX50 Cryostat (Thermo Fisher Scientific). Sections were mounted onto slides, and the slides were fixed using ice-cold acetone for 5 min. Each slide underwent the following procedure for hematoxylin & eosin staining: 1) 2 min wash with de-ionized water, 2) 1 min hematoxylin stain, 3) 2 min water wash, 4) 2 min in 70% ethanol, 5) 2 min in 100% ethanol, 6) 1 min eosin stain, 7) 2 min in 70% ethanol, 8) 2 min in 100% ethanol, and 9) 2 min in xylene. The slides were dried, and coverslips were mounted using Permount Mounting Medium (Thermo Fisher Scientific). Slides were then imaged using an Axioskop 2 microscope (Carl Zeiss).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

All documents referred to in this application are hereby incorporated by reference in their entirety.

What is claimed:

1. A composition comprising: 4-phenylbutyrate (PBA) or a pharmaceutically acceptable salt thereof in an effective concentration as a therapeutic agent against viral infections.

2. The composition of claim 1, wherein the PBA is present as a salt.

3. The composition of claim 2, wherein the PBA is sodium 4-phenylbutanoate.

4. The composition of any one of claims 1-3, wherein the concentration of the PBA or salt thereof ranges from about 1 mM to about 200 mM, or has a cumulative concentration ranging from about 10 mg/kg to about 600 mg/kg.

5. The composition of claim 4, comprising 100 mg/kg of the PBA or salt thereof.

6. The composition of any one of claims 1-5, further comprising a drug delivery agent.

7. The composition of claim 6, wherein the drug delivery agent is selected from a polymeric agent, activated carbon agent, metal agent, metal oxide agent, liposomal agent, peptide/protein agent, sugar agent, and combinations thereof.

8. The composition of any one of claims 1-7, comprising PBA or a salt thereof and a drug delivery agent as a nanoformulation, microformulation, or macroformulation.

9. The composition of any one of claims 1-8, further comprising one or more additional therapeutic agents.

10. The composition of claim 9, wherein the one or more additional therapeutic agent is selected from an antiviral agent, anti-inflammatory agent, pain reducing agent, and combinations thereof.

11. The composition of claim 10, wherein the antiviral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine, or sofobuvir.

12. The composition of claim 11, wherein the antiviral agent is trifluridine.

13. The composition of any one of claims 10-12, comprising 10 mg/kg of the antiviral agent.

14. The composition of claim 10, wherein the anti-inflammatory agent is selected from ibuprofen, naproxen, ketoprofen, tometin, etodolac, flurbiprofen, diclofenac, misoprostol, piroxicam, fenoprofen, indomethacin, sulndac, nabumetone, ketorolac, famotidine, mefenamic acid, diflunisal, meloxicam, and combinations thereof.

15. The composition of claim 10, wherein the pain reducing agent is selected from ibuprofen, diphenhydramine, acetaminophen, magnesium sailicylate, aspirin, meprobamate, ziconotide, butalbitalor, codeine, hydrocodone, dihydrocodone, oxycodone, naloxone, pentazocine, fentanyl, morphine, hydromorphone, buprenorphine, methadone, meperidine, buprenorphine, oxymorphone, tramadol, nalbuphine, propoxyphene, tapentadol, alfentanil, sufentanil, remifentanil, and combinations thereof.

16. The composition of any one of claims 1-15, formulated for ophthalmic administration.

17. The composition of claim 16, comprising an ophthalmic excipient.

18. The composition of claim 17, wherein the ophthalmic excipient is selected from Cyclodextrins, Carbopol or carbomer or acrylic acid polymers, Poloxamers, Xyloglucan, Methylcellulose, Hydroxypropyl Methylcellulose, Ethyl (Hydroxyethyl) Cellulose, Pseudolatexes, Cellulose Acetate Phthalate, Gellan Gum, Alginate, Carrageenans, Hyaluronic Acid, Sodium acetate, Edetate disodium, Hypromellose, Acetic acid, Alcohol, Alginic acid, Amerchol-cab, Antipyrine, Benzalkonium chloride, Benzododecinium bromide, Boric acid, Caffeine, Calcium chloride, Carbomer 1342, Carbomer 934P, Carbomer 940, Carbomer homopolymer type B (allyl pentaerythritol cross-linked), Carboxymethylcellulose sodium, Castor oil, Cetyl alcohol, Chlorobutanol, Citric acid, Citric acid monohydrate, Creatinine, Divinylbenzene styrene copolymer, Ethylene vinyl acetate copolymer, Gellan gum (low acyl), Glycerin, Glyceryl stearate, Hypromelloses, Lanolin, Lauralkonium chloride, Lauroyl sarcosine, Magnesium chloride, Methylparaben, Mineral oil, Nonoxynol-9, Octoxynol-40, Petrolatum, Phenylethyl alcohol, Phenylmercuric acetate, Phenylmercuric nitrate, Polidronium chloride, Poloxamer 188 or 407, Polycarbophil, Polyethylene glycol 400 or 8000, Polyoxyl 35 castor oil, Polyoxyl 40 hydrogenated castor oil, Polyoxyl 40 stearate, Polypropylene glycol, Polysorbate 20, Polyvinyl alcohol, Potassium chloride, Potassium sorbate, Povidone K29/32, Povidone K30, Povidone K90, Povidones, Propylene glycol, Propylparaben, Soda ash, Sodium acetate, Sodium bisulfate, Sodium borate, Sodium borate decahydrate, Sodium carbonate, Sodium chloride, Sodium citrate, Sodium metabisulfite, Sodium nitrate, Sodium sulfate, Sodium sulfite, Sodium thiosulfate, Sorbic acid, Sorbitol, Stabilized oxychloro complex, Sulfuric acid, Thimerosal, Titanium dioxide, Tocophersolan, Trisodium citrate dehydrate, Tromethamine, Tyloxapol, Xanthan gum, Zinc chloride, and combinations thereof.

19. The composition of any one of claims 1-18, formulated for topical administration.

20. The composition of claim 19, comprising a mucosal penetrating agent.

21. The composition of claim 20, wherein the mucosal penetrating agent comprises poloxamer 407.

22. A method of treating a disease or disorder in a subject, comprising administering to the subject a therapeutically effective amount of 4-phenylbutyrate (PBA) or a pharmaceutically acceptable salt thereof.

23. The method of claim 22, wherein the disease or disorder is a viral infection.

24. The method of claim 23, wherein the viral infection is caused by Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, humanherpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCoV NL63, HKU1, 229E and human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simple, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

25. The method of claim 22, wherein the disease or disorder is associated with a herpes simplex virus (HSV).

26. The method of claim 25 wherein the disease or disorder is associated with herpes simplex virus 1 (HSV-1) or herpes simplex virus 2 (HSV-2).

27. The method of claim 25 or 26, wherein the disease or disorder is selected from corneal inflammation, corneal hypertension, corneal sensitivity, corneal dryness, keratitis, ocular herpes, herpes gingivostomatitis, herpes labialis, herpes genitalis, herpetic whitlow, herpes gladiatorum, herpesviral encephalitis, herpesviral meningitis, herpes esophagitis, neonatal herpes simplex, herpetic sycosis, eczema herpeticum, herpetic keratoconjunctivitis, herpetic uveitis, Sjögren's syndrome, dry eye disease, and combinations thereof.

28. The method of any one of claims 22-27, comprising administering to a patient a therapeutically effective amount of a composition comprising PBA or a pharmaceutically acceptable salt thereof, and one or more additional therapeutic agents.

29. The method of claim 28, wherein the one or more additional therapeutic agent is selected from an antiviral agent, anti-inflammatory agent, pain reducing agent, and combinations thereof.

30. The method of claim 29, wherein the antiviral agent is Aciclovir or a nucleoside analog thereof, maraviroc, enfuvirtide, amantadine, lamivudine, nevirapine, efavirenz, dolutegravir, elvitegravir, raltegravir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine, or sofobuvir.

31. The method of claim 30, wherein the antiviral agent is trifluridine.

32. The method of any one of claims 22-31, comprising parenteral, nasal, oral, pulmonary, topical, vaginal, rectal, ophthalmic or sublingual administration.

33. The method of any one of claims 22-32, comprising prophylactic or therapeutic administration.

34. The method of any one of claims 22-33, comprising administration via preloaded syringe.

35. The method of any one of claims 22-34, comprising administration via preloaded dermal (skin) patch.

36. A method of treating an ocular disease or condition in a subject in need thereof, comprising administering to the subject 4-phenylbutyrate (PBA) or a pharmaceutically acceptable salt thereof.

37. The method of claim 36, comprising administering to a patient a therapeutically effective amount of a composition comprising PBA or a pharmaceutically acceptable salt thereof, and one or more additional therapeutic agents.

38. The method of claim 36 or 37, wherein the ocular disease or condition is selected from corneal inflammation, corneal hypertension, corneal sensitivity, corneal dryness, ocular herpes, herpetic keratoconjunctivitis, herpetic uveitis, Sjögren's syndrome, keratitis, and combinations thereof.
